SEQUENCE LISTING (18 sequences)

SEQ ID NO: 1 - protein, 398 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVTLELELPESPVNQDLGMFLVTISCYTRGGRIISTSSRSVMLHYRSDLLQMLDTLVFSSLLLFGFAEQKQLLEVELYADYRENSYVPTTGAIIEIHSKRIQLYGAYLRIHAHFTGLRYLLYNFPMTCAFIGVASNFTFLSVIVLFSYMQWVWGGIWPRHRFSLQVNIRKRDNSRKEVQRRISAHQPGPEGQEESTPQSDVTEDGESPEDPSGTEGQLSEEEKPDQQPLSGEEELEPEASDGSGSWEDAALLTEANLPAPAPASASAPVLETLGSSEPAGGALRQRPTCSSS

SEQ ID NO: 2 - protein, 74 aa, Homo sapiens
MVNDPPVPALLWAQEVGQVLAGRARRLLLQFGVLFCTILLLLWVSVFLYGSFYYSYMPTVSHLRDCAFLLQDRL

SEQ ID NO: 3 - protein, 110 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLKCMDSRIVLP

SEQ ID NO: 4 - protein, 111 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPSCYLRA

SEQ ID NO: 5 - protein, 110 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPCYLRA

SEQ ID NO: 6 - protein, 112 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVNLRA

SEQ ID NO: 7 - protein, 137 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVTLELELPESPVNQDLGMFLVTISCYTRGG

SEQ ID NO: 8 - protein, 350 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVMLHYRSDLLQMLDTLVFSSLLLFGFAEQKQLLEVELYADYRENSYVPTTGAIIEIHSKRIQLYGAYLRIHAHFTGLRYLLYNFPMTCAFIGVASNFTFLSVIVLFSYMQWVWGGIWPRHRFSLQVNIRKRDNSRKEVQRRISAHQPGPEGQEESTPQSDVTEDGESPEDPSGTEGQLSEEEKPDQQPLSGEEELEPEASDGSGSWEDAALLTEANLPAPAPASASAPVLETLGSSEPAGGALRQRPTCSSS

SEQ ID NO: 9 - protein, 398 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVTLELELPESPVNQDLGMFLVTISCYTRGGRIISTSSRSVMLHYRSDLLQMLDTLVFSSLLLFGFAEQKQLLEVELYADYRENSYVPTTGAIIEIHSKRIQLYGPYLRIHAHFTGLRYLLYNFPMTCAFIGVASNFTFLSVIVLFSYMQWVWGGIWPRHRFSLQVNIRKRDNSRKEVQRRISAHQPGPEGQEESTPQSDVTEDGESPEDPSGTEGQLSEEEKPDQQPLSGEEELEPEASDGSGSWEDAALLTEANLPAPAPASASAPVLETLGSSEPAGGALRQRPTCSSS

SEQ ID NO: 10 - protein, 231 aa, Homo sapiens
MVNDPPVPALLWAQEVGQVLAGRARRLLLQFGVLFCTILLLLWVSVFLYGSFYYSYMPTVSHLSPVHFYYRTDCDSSTTSLCSFPVANVSLTKGGRDRVLMYGQPYRVTLELELPESPVNQDLGMFLVTISCYTRGGRIISTSSRSVMLHYRSDLLQMLDTLVFSSLLLFGFAEQKQLLEVELYADYRENSYVPTTGAIIEIHSKRIQLYGATSASTRTSLGSDTCYTTSR

SEQ ID NO: 11 - protein, 15 aa, Homo sapiens
PVPALLWAQEVGQVL

SEQ ID NO: 12 - protein, 17 aa, Homo sapiens
PEDPSGTEGQLSEEEKP

SEQ ID NO: 13 - protein, 12 aa, Homo sapiens
EIHSKRIQLYGA

SEQ ID NO: 14 - DNA, 1664 bp, Homo sapiens
ctgaatcttcgtttctctcccagggaccctccattttccatatccaggaaaatgtgatgcgccacaggtatcagcgtctggatcgccacttcacgttttagccacaagtgactcagtggaagatccagagtcaacagaggctcgtcaggaagatgtctacagaaaaggtagaccaaaaggaggaagctggggaaaaagaggtgtgcggagaccagatcaaaggaccggacaaagaggaggaaccaccagctgctgcatcccatggccaggggtggcgtccaggtggcagagcagctaggaacgcaaggcctgaacctggggccagacaccctgctctcccggccatggtcaacgaccctccagtacctgccttactgtgggcccaggaggtgggccaagtcttggcaggccgtgcccgcaggctgctgctgcagtttggggtgctcttctgcaccatcctccttttgctctgggtgtctgtcttcctctatggctccttctactattcctatatgccgacagtcagccacctcagccctgtgcatttctactacaggaccgactgtgattcctccaccacctcactctgctccttccctgttgccaatgtctcgctgactaagggtggacgtgatcgggtgctgatgtatggacagccgtatcgtgttaccttagagcttgagctgccagagtcccctgtgaatcaagatttgggcatgttcttggtcaccatttcctgctacaccagaggtggccgaatcatctccacttcttcgcgttcggtgatgctgcattaccgctcagacctgctccagatgctggacacactggtcttctctagcctcctgctatttggctttgcagagcagaagcagctgctggaggtggaactctacgcagactatagagagaactcgtacgtgccgaccactggagcgatcattgagatccacagcaagcgcatccagctgtatggagcctacctccgcatccacgcgcacttcactgggctcagatacctgctatacaacttcccgatgacctgcgccttcataggtgttgccagcaacttcaccttcctcagcgtcatcgtgctcttcagctacatgcagtgggtgtgggggggcatctggccccgacaccgcttctctttgcaggttaacatccgaaaaagagacaattcccggaaggaagtccaacgaaggatctctgctcatcagccagggcctgaaggccaggaggagtcaactccgcaatcagatgttacagaggatggtgagagccctgaagatccctcagggacagagggtcagctgtccgaggaggagaaaccagatcagcagcccctgagcggagaagaggagctagagcctgaggccagtgatggttcaggctcctgggaagatgcagctttgctgacggaggccaacctgcctgctcctgctcctgcttctgcttctgcccctgtcctagagactctgggcagctctgaacctgctgggggtgctctccgacagcgccccacctgctctagttcctgaagaaaaggggcagactcctcacattccagcactttcccacctgactcctctcccctcgtttttccttcaataaactattttgtgtcagcttcaaaaaaaaaaaaaaaaaaaaaaaaaaaaaaa

SEQ ID NO: 15 - DNA, 20 bp, Artificial sequence
  Artificial sequence descriptionprimer
gattgagcccagaagttcca

SEQ ID NO: 16 - DNA, 21 bp, Artificial Sequence
  Artificial Sequence Descriptionprimer
ccagatgattaagccagttgc

SEQ ID NO: 17 - DNA, 20 bp, Artificial sequence
  Artificial sequence descriptionprimer
cccctgcagtggagtctgta

SEQ ID NO: 18 - DNA, 20 bp, Artificial sequence
  Artificial sequence descriptionprimer
agtcaggtgggaaagtgctg

(19) United States
(12) Patent Application Publication
(10) Pub. No.: US 2005/0130916 A1
(43) Pub. Date: Jun. 16, 2005

(54) Nucleic acid coding for the cgl1 polypeptide and diagnostic and therapeutic application of said nucleic acid and of the cgl1 polypeptide

(76) Inventors: Jocelyne Magre, Paris (FR); Jacqueline Capeau, Saint-Mande (FR); Mark Lathrop, Paris (FR); Marc Delepine, Paris (FR)

Correspondence Address: JACOBSON HOLMAN PLLC, 400 SEVENTH STREET N.W., SUITE 600, WASHINGTON, DC, 20004, US

(21) Appl. No.: 10/475,856

(22) PCT Filed: May 3, 2002
(86) PCT No.: PCT/FR02/01541

(30) Foreign Application Priority Data
May 4, 2001 (FR) 01/06037

(51) Int. Cl. C12Q 1/68; C07H 21/04; A61K 48/00; C07K 14/47

(52) U.S. Cl. 514/44; 536/23.5; 435/6; 435/69.1; 435/320.1; 435/325; 530/350

(57) ABSTRACT

The invention provides a nucleic acid coding for the CGL1 polypeptide with the SEQ ID No 1 amino acid sequence or also fragments or variants of the CGL1 polypeptide. The invention also relates to the use of a nucleic acid such as defined hereinabove for producing a nucleotide probe or primer specific for the normal cgl1 gene or the mutated cgl1 gene. Another object of the invention is also to provide methods for screening a candidate compound interacting with the CGL1 polypeptide or modulating the expression of the cgl1 gene as well as sets or kits for screening such candidate compounds.

The present invention relates to the field of prevention and treatment of pathologies such as lipodystrophias, more particularly congenital generalized lipodystrophia (CGL) as well as diabetes (of type 1 and type 2).

Lipodystrophias are defined as distribution alterations in the body adipose tissue which can be reduced or increased. They include obesity and the metabolism syndrome (or insulin-resistance syndrome or X syndrome).

### STATE OF THE ART

Congenital generalized lipodystrophia (CGL) has been described for the first time (in the fifties) by BERARDINELLI (1954) and SEIP (1959). Such pathology has been reviewed in detail by SEIP in 1996.

Generalized lipodystrophia (GL) belongs to the heterogeneous group of syndromes wherein distribution alterations in the body fat depots as well as an insulin resistance are simultaneously present.

Lipodystrophia syndromes have been classified depending on the lipoatrophy extent, whether it is local or generalized. In generalized lipodystrophias (GL), a severe depletion is observed of both subcutaneous adipose tissue and visceral adipose tissue. Both GL subtypes have been distinguished depending on the appearance age of the lipoatrophy. The first subtype is the congenital generalized lipodystrophia (CGL), also referred to as [symbol] BERARDINELLI-SEIP syndrome [symbol] wherein a lack of adipose tissue is observed from birth or early from early childhood. The second type is referred to as acquired generalized lipodystrophia or also [symbol] LAWRENCE syndrome [symbol] wherein the lipoatrophy is only observed during childhood or adult age. Based on the observation of an incidence both in man and woman, on the frequent recurrence of the pathology in the offspring, as well as on the observation of a parental consanguinity rate up to 50% in the affected patients, the transmission of the CGL trait in a recessive autosomal manner was considered as being quite probable.

A marker segregation study conducted in six CGL affected Norwegian families has made it possible to observe, in two of those families, a heterochromatic phenomenon on the 9q branch of chromosome 9 (GEDDE-DAHL, 1996). In such a study, the researchers had explicitly excluded the hypothesis of an allelic association between the APOAI1 marker, located on the 11q 23.3 locus of chromosome 11, and the causal genetic determinant for CGL.

Another study had also located the CGL causal genetic determinant on chromosome 9. Thus, GARG et al. (1999), conducting a positional cloning study, identified in most of the families a locus associated to CGL on 9q34 chromosome, within a 8,7 cM interval respectively bordered by D9S1818 and D9S1826 markers. However, those authors did not identify the causal gene in such a 8,7 cM interval.

Currently, few treatments for the CGL affected patients are available. The most important part of the present treatments consist in providing the patients with the optimum quality and quantity of energy necessary for their immediate metabolism. But it is very difficult for the patients to follow a sufficiently strict diet for their whole life. Despite administering high insulin doses in order to overcome the insulin resistance observed upon CGL, the diabetes control remains poor and the patients develop serious diabetic complications.

Usage has also been made of pimozide, a dopamine blocking drug, because of the observation of increased amounts of the hypophyseal hormone releasing factors in the blood of CGL affected patients. Fenfluramin has also been used, which has an anorectic effect and more particularly reduces the easily digestible carbohydrates consumption. It is a serotoninergic agonist compound increasing the serotonin rate in the CNS. However, administering fenfluramin was followed by numerous unwanted effects, including drowsiness, diarrhea and dry mouth. IGF-1 has also been used. However, IGF-1 is a substance with a high anabolic power and which is known for stimulating the cell growth in some tissues. IGF-1 is therefore able to dangerously deteriorate a cardiomyopathy in the thus treated CGL affected patients.

There is therefore a need in the present knowledge state for efficient means adapted for preventing or treating lipodystrophias, in particular CGL, that are able to be used in the long term with patients and leading to the as reduced as possible unwanted effects. Such specific preventive or curative means could only be developed in a targeted way without having previously identified the causal genetic determinant for this syndrome.

### SUMMARY OF THE INVENTION

The Applicant has presently identified a causal gene for the congenital generalized lipodystrophia (CGL) syndrome, located on the 11q13 locus of the human chromosome 11. The CGL causal gene has been referred to as cgl1 by the Applicant, who also identified the protein coded by such a gene, referred to as CGL1.

The pathologies being preferably aimed at according to the invention are as follows:
generalized congenital lipodystrophia, or lipoatrophic diabetes, or Berardinelli-Seip syndrome or Berardinelli-Seip congenital lipodystrophia,
Lawrence syndrome,
acquired lipodystrophia or secondary lipodystrophia,
partial lipodystrophia,
secondary (or acquired) lipodystrophya to the antiretroviral treatment of HIV infected patients,
type 1 diabetes,
type 2 diabetes,
obesity,
metabolic syndrome or insulin-resistant syndrome to the X syndrome.

According to the invention, the cgl1 gene may also be referred to as [symbol] BSCL2 [symbol] .

According to the invention, the CGL1 protein may also be referred to as [symbol] Seipine [symbol] .

It has therefore been provided according to the invention a nucleic acid coding for CGL1 polypeptide of an amino acid sequence SEQ ID No 1 or also fragments or variants of CGL1 polypeptide.

The invention also relates to the use of a nucleic acid such as defined herein above for manufacturing a specific probe or nucleotide primer for the normal cgl1 gene or the mutated cgl1 gene.

The invention also relates to methods for detecting a mutation within a nucleic acid coding for CGL1 polypeptide, said methods implementing one or more specific nucleotide probes or primers for the mutated cgl1 gene, as well as sets or kits for detecting a mutation within the cgl1 gene.

The invention also relates to recombinant vectors comprising a nucleic acid coding for CGL1 polypeptide, more particularly recombinant vectors useful in genic therapy methods, as well as recombinant host cells being transfected or transformed by such a nucleic acid or by a vector such as defined herein above.

Another object of the invention is the use of a nucleic acid coding for CGL1 polypeptide for manufacturing a drug for preventing or treating a lipodystrophia, a diabetes or an obesity, and in particular a lipodystrophia associated with the CGL syndrome.

It also relates to pharmaceutical compositions for preventing or treating a lipodystrophia or a diabetes, characterized in that they comprise a nucleic acid coding for CGL1 polypeptide or a recombinant vector or a recombinant host cell as defined herein above, in association with one or more physiologically compatible excipients.

Another object of the invention is also the CGL1 polypeptide with a sequence SEQ ID No 1 or also a fragment or variant of the CGL1 polypeptide.

It also relates to the use of a CGL1 polypeptide for manufacturing a drug for preventing or treating a lipodystrophia or a diabetes as well as a pharmaceutical composition comprising a CGL1 polypeptide.

The invention also relates to an antibody raised against the CGL1 polypeptide, or also a fragment or a variant of the CGL1 polypeptide, as well as detection methods and sets or kits implementing such an antibody.

Another object of the invention is also to provide methods for screening a candidate compound interacting with the CGL1 polypeptide as well as sets or kits for screening such candidate compounds.

It also relates to methods for screening a candidate compound modulating the expression of the cgl1 gene as well as to sets or kits for screening such candidate compounds.

### DESCRIPTION OF THE FIGURES

FIG. 1.

FIG. 1A shows the CGL families of two geographically distinct groups, respectively Lebanese and Norwegian, which have been studied in the linking analysis conducted on the whole genome. The solid symbols represent the affected individuals and the empty symbols represent the unaffected individuals. The pedigrees have been simplified and only show the affected offspring that has been effectively genotyped. In the CGL-01 group, the individual represented with a star (*) has the same name as the last three families of the pedigree respecting the recessive transmission mode of the disease for patients #4. For 11q 13 chromosome, the haplotype analysis is schematically represented by the bars located under the genotyped individuals: the solid bars correspond to the respective bearing allele, which segregates with the disease and shows a [symbol] funding [symbol] effect in each population. The numbering of the affected individuals corresponds to that used in FIG. 2, wherein details of fine mapping are shown.

FIG. 1B shows a multipoint analysis of the Lebanese and Norwegian families at the 11q13 locus. The marker location is indicated at the bottom of the diagram. The Iod score of 15.2 indicates that the locus of the CGL disease is located in the D1154191-D115997 interval.

FIG. 2 shows the homozygosity mapping at the 11q13 locus. The genotypes are shown for the patients after evaluation of the haplotypes in each family from Lebanon or from Norway. The italic characters represent the genotypes for patients 4, 12, 13, 24 and 25, which have been inferred from other family relatives, due to the DNA absence for the study; x represents a haplotype to the disease unidentified in the available DNA's. The microsatellite markers are indicated in the left column; the arrows indicate the markers used for the mapping of the whole genome. The solid boxes indicate the homozygosity regions.

FIG. 3 shows the haplotype analysis in families from various ethnic origins being co-segregant at the 11q13 locus. The individuals have been genotyped with six markers covering an approximately 10 cM interval. For each family, the ethnic origin is indicated when it differs from the living country. In the CGL-16 family, the letter d as a genotype to the CA10 marker corresponds to a deletion.

FIG. 4A shows deletions in the cgl1 gene, a human homologue of the Gng31g murine gene. The eleven exons have been amplified in the form of seven fragments in each member of the GCL-16 consanguineous family, including the affected children (monozygotic twins).

FIG. 4B shows the distribution of mutations within the cgl1 gene. Mutations are represented as a function of their type: wide deletion (−), a false sense mutation (O), a nonsense mutation (X), mutation in the splicing site (I), mutation of reading framework change due to an insertion (♦), a deletion (♦) or a deletion/insertion (⋄).

FIG. 5 shows the expression profile of the cgl1 gene.

FIG. 5A is an autoradiographic photograph showing the hybridization of a cDNA probe covering exons 1 to 11 versus mRNA poly(A)⁺ gels (about 2 μg) of various tissues, showing the existence of transcription products being respectively of about 2,4 kb, about 1,8 kb and about 2 kb.

FIG. 5B shows autoradiography photographs showing the hybridization of a cDNA probe covering exons 1 to 11 on a gel containing total human RNA (20 μg) from the abdominal and visceral subcutaneous tissues, as compared to brain, heart and liver tissues.

FIG. 5C shows the quantification in the autoradiography photographs obtained after hybridization of a cDNA probe covering exons 1 to 11 on poly(A)⁺ RNA of various tissues. The results show that the highest expression levels have been observed for brain and testicles.

FIG. 6 shows an alignment of the sequences of the CGL1 protein with respectively Gng31g protein of mus musculus and CG9904 protein of Drosophila melanogaster. The preserved amino acid residues are represented in the solid boxes. The putative transmembrane fields are underlined.

### GENERAL DEFINITIONS

As used herein the term “isolated” refers to a biological material (nucleic acid or protein) which has been removed from its original environment (the environment where it naturally occurs).

For example a polynucleotide naturally occurring in a plant or an animal is not isolated. The same polynucleotide separated from adjacent nucleic acids within which it is naturally inserted in the genome of the plant or the animal is considered as “isolated”.

Such a polynucleotide could be included into a vector and/or such a polynucleotide could be included into a composition and still remain in an isolated state because the vector or the composition is not its natural environment.

The term “purified” does not require that the material should be present in an absolute purity form, excluding the presence of other compounds. It is rather a relative definition.

A polynucleotide is in a “purified” state after purification of the starting material or the raw material of at least one order of magnitude, preferably 2 to 3 and more preferably 4 or 5 orders of magnitude.

For the purpose of the present disclosure, the expression “nucleotide sequence” may be used for indiscriminately referring to a polynucleotide or a nucleic acid. The expression “nucleotide sequence” includes the genetic material itself and is therefore not limited to the information relating to its sequence.

The expressions “nucleic acid”, “polynucleotide”, “oligonucleotide” as well as “nucleotide sequence” include RNA, DNA, cDNA sequences as well as RNA/DNA hybrid sequences of more than one nucleotide, indiscriminately under the single chain form or under the duplex form.

The term “nucleotide” refers to both natural nucleotides (A, T, G, C) and modified nucleotides comprising at least one modification such as (1) a purine analogue, (2) a pyrimidine analogue or (3) an analogous sugar, some examples of such modified nucleotides being disclosed for example in PCT Application No WO 95/04 064.

For the purpose of the present invention, a first polynucleotide is considered as being “complementary” to a second polynucleotide when each base of the first nucleotide is paired with the complementary base of the second polynucleotide having the reverse orientation. The complementary bases are A and T (or A and U), or C and G.

The term “variant” for a nucleic acid according to the invention refers to a nucleic acid differing of one or more bases from the reference polynucleotide. A nucleic acid variant may be of natural origin, such as a naturally occurring allelic variant or may be also a non natural variant obtained for example with mutagenesis techniques.

Generally, the differences between the reference nucleic acid and the nucleic acid variant are small so that the nucleotide sequences of the reference nucleic acid and the nucleic acid variant are very close and, in numerous regions, are identical. The nucleotide modifications present in a nucleic acid variant may be silent, meaning that they do not alter the amino acid sequences coded by said nucleic acid variant.

However, the nucleotide changes within a nucleic acid variant may also result in substitutions, additions, deletions in the polypeptide coded by the nucleic acid variant compared to the peptides coded by the reference nucleic acid. Additionally, nucleotide modifications in the coding regions may produce substitutions, either conservative or not, in the amino acid sequences.

Preferably, the nucleic acid variants according to the invention code for polypeptides that substantially maintain the same biological function or activity as the polypeptide of the reference nucleic acid or the ability to be recognised by antibodies raised against the polypeptides coded by the initial nucleic acid.

Some nucleic acid variants will thus code for mutated forms of polypeptides the systematic study of which will enable to infer structure/activity relationship of the subject proteins.

The term “fragment” as used herein refers to a reference nucleic acid according to the invention, a nucleotide sequence having a reduced length compared to the reference nucleic acid and comprising, on the common part, a nucleotide sequence identical to the reference nucleic acid.

Such a nucleic acid “fragment” according to the invention may be, if need be, included in a larger polynucleotide of which it is a component.

Such fragments comprise, or alternatively consist of, oligonucleotides with a length ranging from 8, 10, 12, 15, 18, 20 to 25, 30, 40, 50, 70, 80, 100, 200, 500, 1000 or 1500 consecutive nucleotides in a nucleic acid according to the invention.

The “variant” of a polypeptide according to the invention primarily refers to a polypeptide the amino acid sequence of which contains one or more substitutions, additions or deletions of at least one amino acid residue compared to the amino acid sequence of the reference polypeptide, being well understood that the amino acid substitutions may indiscriminately be conservative or not.

Preferably, a [symbol] variant [symbol] of a polypeptide according to the invention has a N-terminal amino acid sequence identical to the reference polypeptide. Thus, the preferred variants of the CGL1 polypeptide have a N-terminal sequence starting with the amino acid sequence [symbol] Met-Val-Asn-Asp-Pro [symbol] or [symbol] Met-Val-Asn-Asp-Pro-Pro-Val-Pro-Ala [symbol] .

The “fragment” of a polypeptide as used herein will refer to a polypeptide with an amino acid sequence shorter than that of the reference polypeptide and comprising on the common part with those reference polypeptides, an identical amino acid sequence.

Such fragments may, if need be, be included within a larger polypeptide of which they are part.

Such fragments of a polypeptide according to the invention may have a length of 10, 15, 20, 30 to 40, 50, 100, 200 or 300 amino acids.

The “identity percentage” between two nucleotide or amino acid sequences, as used herein, can be determined comparing two optimally aligned sequences, through a comparison window.

The part of the nucleotide sequence or polypeptide within the comparison window may thus comprise additions or deletions (for example “gaps”) compared to the reference sequence (which does not include such additions or deletions) so as to obtain an optimal alignment of both sequences.

The percentage is calculated determining the number of positions where an identical nucleic base or amino acid residue is observed for both sequences (nucleic or peptidic) to be compared, followed by dividing the number of positions where there is an identity between both bases or amino acid residues by the total number of positions within the comparison window, and then by multiplying the result by 100 so as to obtain the sequence identity percentage.

The optimal alignment of the sequences for the comparison may be achieved with a computer, using known algorithms contained in the package from the WISCONSIN GENETICS SOFTWARE PACKAGE corporation, GENETICS COMPUTER GROUP (GCG), 575 Science Doctor, Madison, Wis.

By way of illustration, the sequence identity percentage could be achieved using the BLAST software (versions BLAST 1.4.9 dated March 1996, BLAST 2.0.4 dated February 1998 and BLAST 2.0.6 September 1998), exclusively using the default parameters (S. F Altschul et al., J. Mol. Biol. 1990 215:403-410, S. F Altschul et al., Nucleic Acids Res. 1997 25:3389-3402). Blast searches for similar/homologous sequences to a reference “request” sequence, using the Altschul et al. algorithm. The request sequence and the data bases to be used may be peptidic or nucleic, any combination being possible.

The expression “strongly stringent hydridization conditions” as used herein refers to the following conditions:
1. Competition of the membranes and PRE HYBRIDIZATION
   Mix 40 μl salmon sperm DNA (10 mg/ml)+40 μl human placental DNA (10 mg/ml)
   Denature 5 min at 96° C., then immerge the mixture into ice
   Remove the 2× SSC and pour 4 ml formamide mix in the hybridization tube containing the membranes
   Add the mixture of both denatured DNAs
   Incubate at 42° C. for 5 to 6 hours while spinning.
2. Competition of the labeled probe
   Add to the labeled and purified probe 10 to 50 μl DNA Cot I according to the repeat quantity
   Denature 7 to 10 min at 95° C.
   Incubate at 65° C. for 2 to 5 hours.
3. HYBRIDIZATION
   Remove the pre-hybridization mix
   Mix 40 μl salmon sperm DNA+40 μl human placental DNA; denature 5 min at 96° C., then immerge into ice
   Add into the hybridization tube 4 ml formamide mix, the mixture of both DNA and the labeled/DNA Cot I denatured probe.
   Incubate 15 to 20 hrs at 42° C. while spinning.
4. Washing Operations
   One washing operation at room temperature in 2× SSC, so as to rinse.
   Twice 5 minutes at room temperature 2× SSC and 0.1% SDS at 65° C.
   Twice 15 minutes at 65° C. 1× SSC and 0.1% SDS at 65° C.
      Wrap the membranes in Saran and expose them.

The above-described hybridization conditions are adapted to the hybridization in highly stringent conditions of a nucleic acid molecule with a length varying from 20 nucleotides to several hundreds of nucleotides.

It is understood that the above described hybridization conditions can be adapted depending on the length of the nucleic acid, the hybridization of which is required, or on the selected marking type, depending on the techniques known to the man of the art.

The conditions appropriate for the hybridization may for example be adapted according to the teaching contained in the work by HAMES and HIGGINS (1985) or also in the work by F. AUSUBEL et al. (1989).

### DETAILED DESCRIPTION OF THE INVENTION

The inventors have made a marker association mapping and a homozygosity mapping in individuals from nine CGL affected families coming from two distinct geographical isolates, respectively from Lebanon and Norway, which enabled them to isolate one of the causal genes for the congenital generalized lipodystrophia syndrome in a small region of the 11q13 chromosome locus.

The inventors have conducted a first association investigation on the whole genome, the results of which showed that two adjacent markers, respectively the D11S4191 and D11S987 markers located on the 11q13 chromosome locus were homozygotic in most of the patients. An association analysis with two additional markers, respectively the D11S1765 and D11S1883 markers located inside the above-mentioned interval, confirmed the first observations and showed the presence of identical alleles in eighteen patients of the Lebanese families for the D11S1883 marker.

The inventors then characterized 25 additional microsatellite markers, twelve of which are located between the D11S4191 and D11S987 markers. A second association investigation conducted with such markers has make it possible to determine that the locus bearing the CGL causal genetic determinant is located in a region of approximately 2,5 Mb located between the D11S4076 and D11S480 markers. Some of the patients included in the investigation are homozygotic for a null allele corresponding to the CA10 marker. This result made it possible for the inventors to identify a congenital generalized lipodystrophia causal gene amongst the 27 genes studied in the D11S4076-D11S480 interval. Such a gene is obtained in the DNA insert of a BAC vector referred to as RP11-83H9 having the partial nucleotide sequences referenced in the GenBanK data base under the access number No AP001458.

The CGL causal gene has been called [symbol] cgl1 gene [symbol] by the inventors.

Through sequencing the cgl1 gene in CGL affected patients, the inventors have identified numerous mutations inducing the coding for the mutated CGL1 polypeptides, more particularly the truncated CGL1 polypeptides.

The identification by the inventors of the CGL1 gene, responsible for the congenital generalized lipodystrophia syndrome (CGL), makes it possible for them to develop detection tools for the normal cgl1 gene and for the mutated cgl1 gene amongst patients, expression tools for the normal cgl1 gene in order to complement the expression lack of such a gene in patients, preferably CGL patients, CGL preventive or curative means as well as screening tools for pharmaceutically efficient compounds for preventing or treating various pathologies and preferably pathologies listed in the [symbol] SUMMARY [symbol] section herein above.

Thus, a first object of the invention is a nucleic acid coding for the CGL1 polypeptide with a amino acid sequence SEQ ID No 1 or a fragment or a variant of the CGL1 polypeptide.

Generally, the nucleic acids according to the present invention are of an isolated or purified form.

Genomic Nucleic Acids Coding for the CGL1 Polypeptide

As previously set forth, the genomic sequence of the cgl1 gene is entirely contained within the DNA insert of the BAC vector referred to as RT11-831H9, non organised sequences of which are referenced under the access no AP001458 in the GenBank data base.

The inventors have determined that the cgl1 gene comprises 11 exons and 10 introns.

On the basis of the nucleotide numbering of genomic sequence no AP001458 from GenBank, the sequences of the various exons of the cgl1 gene were determined as follows.

The exon 1, which does not comprise any open reading framework, corresponds to the complementary nucleotide sequence to the sequence up to the nucleotide in position 35294 of the AP001458 sequence.

The exon 2 is the nucleotide sequence ranging from the nucleotide in position 161282 up to the nucleotide in position 161598 of the AP001458 sequence. The base A of the initiation codon for the translation is located in position 161387.

The exon 3 is the nucleotide sequence ranging from the nucleotide in position 158346 up to the nucleotide in position 158427 of the AP001458 genomic sequence.

The exon 4 is the complementary sequence to the nucleotide sequence ranging from the nucleotide in position 153740 up to the nucleotide in position 153597 of the AP001458 genomic sequence.

The exon 5 is the complementary sequence to the nucleotide sequence ranging from the nucleotide in position 66927 up to the nucleotide in position 66793 of the AP001458 genomic sequence.

The exon 6 is the complementary sequence to the nucleotide sequence ranging from the nucleotide in position 66603 up to the nucleotide in position 66506 of the AP001458 genomic sequence.

The exon 7 is the complementary sequence to the nucleotide sequence ranging from the nucleotide in position 65551 up to the nucleotide in position 65410 of the AP001458 genomic sequence.

The exon 8 is the complementary sequence to the nucleotide sequence ranging from the nucleotide in position 65271 up to the nucleotide in position 65,205 of the AP001458 genomic sequence.

The exon 9 is the complementary sequence to the nucleotide sequence ranging from the nucleotide in position 64997 up to the nucleotide in position 64917 of the AP001458 genomic sequence.

The exon 10 is the complementary sequence to the nucleotide sequence ranging from the nucleotide in position 64822 up to the nucleotide in position 64742 of the AP001458 genomic sequence.

The exon 11 is the complementary sequence to the nucleotide sequence ranging from the nucleotide in position 64651 of the AP001458 genomic sequence. The base A of the TGA stop codon for the translation of the cgl1 gene is located at the nucleotide 64497 of the AP001458 genomic sequence.

Table 1 hereunder shows the intron-exon linking sequences of the cgl1 gene.

TABLE 1
Intron-exon links of the cgl1 gene

| | | | |
|---|---|---|---|
| | Size | | |
| | (bp) | | |
| Exon | ND | Splicing site 3′ | Splicing site 5′ |

| | | | |
|---|---|---|---|
| 1 | 317 | | CAAAGAGGAGgtaggggcccttgtg |
| 2 | 82 | accctctctttccagGAACCACCAG | TCTACTACAGgtgagaggggccttc |
| 3 | 144 | tccaatctcatttagGACCGACTGT | ACGTGATCGGgtgagtatgggaact |
| 4 | 135 | ggctacctttggcagGTGCTGATGT | TTCGCGTTCGgtaagtgttggtggc |
| 5 | 98 | tcccacccccggcagGTGATGCTGC | AGAGAACTCGgtgagtgaggtgaac |
| 6 | 142 | cccaccccctcacagTACGTGCCGA | CTGGGCTCAGgtgaggggccaactg |
| 7 | 67 | tacttcctgcctcagATACCTGCTA | CTCTTTGCAGgtcgaaaggggcaag |
| 8 | 81 | gtttttcctccacagGTTAACATCC | CATCAGCCAGgtaaaggtgttggct |
| 9 | 81 | tctttggaggtgcagGGCCTGAAGG | TCAGGGACAGgtatggcgcagccac |
| 10 | >155 | cccttttggctgcagAGGGTCAGCT | GCCAGTGATGgtgaggggcatcctg |
| 11 | | cctttcctggcccagGTTCAGGCTC | |

Based on the nucleotide numbering of a second genomic sequence, available in the GenBank data base under number AC 090306 (gi 14595862, clone RP11683Hg), the sequences of the various exons of the cgl1 gene were determined as follows.

The exon 1, that does not comprise any open reading framework, corresponds to the nucleotide sequence ranging from the nucleotide in position 134 035 up to the nucleotide in position 134 273 of the AC 090306 sequence.

The exon 2 is the nucleotide sequence ranging from the nucleotide in position 135 765 up to the nucleotide in position 136 081 of the AC 090306 sequence.

The exon 3 is the nucleotide sequence ranging from the nucleotide in position 138 833 up to the nucleotide in position 138 914 of the AC 090306 genomic sequence.

The exon 4 is the nucleotide sequence ranging from the nucleotide in position 146 670 up to the nucleotide in position 146 813 of the AC 090306 genomic sequence.

The exon 5 is the nucleotide sequence ranging from the nucleotide in position 148 584 up to the nucleotide in position 148 718 of the AC 090306 genomic sequence.

The exon 6 is the nucleotide sequence ranging from the nucleotide in position 148 908 up to the nucleotide in position 149 005 of the AC 090306 genomic sequence.

The exon 7 is the nucleotide sequence ranging from the nucleotide in position 149 960 up to the nucleotide in position 150 101 of the AC 090306 genomic sequence.

The exon 8 is the nucleotide sequence ranging from the nucleotide in position 150 240 up to the nucleotide in position 150 306 of the AC 090306 genomic sequence.

The exon 9 is the nucleotide sequence ranging from the nucleotide in position 150 514 up to the nucleotide in position 150 594 of the AC 090306 genomic sequence.

The exon 10 is the nucleotide sequence ranging from the nucleotide in position 150 689 up to the nucleotide in position 150 769 of the AC 090306 genomic sequence.

The exon 11 is the nucleotide sequence ranging from the nucleotide in position 150 860 up to the nucleotide in position 151 106 of the AC 090306 genomic sequence.

The marker according to the invention referred to as [symbol] CA10 [symbol] is located from the nucleotide in position 145 136 up to the nucleotide in position 145 016 of the AC 090306 genomic sequence.

Given the description of all the structural features of the cgl1 gene, the man of the art will be able to access to all the genomic nucleic acid coding for the cgl1 gene using common molecular biology techniques. In particular, the cgl1 gene may be isolated by the man of the art from the BAC vector referred to as RP11-83H9 and referenced under the access No AP00 1458 and AC 090 306 of the GenBanK data base.

Advantageously, the man of the art can again isolate the genomic nucleic acid of the cgl1 gene from a sample of human DNA, using the primer pair of the respective sequences SEQ ID No 17 and SEQ ID No 18.

The invention relates to a nucleic acid coding for the CGL1 polypeptide characterized in that it comprises the cgl1 gene contained in the BAC vector referred to as RP-11-83H9, the partial nucleotide sequences of which are referenced in the GENBANK data base under the access number no AP001458 or the nucleotide sequences thereof being referenced in the GenBanK data base under No D4ACC7S AC 090 306.

cDNA Coding for the CGL1 Polypeptide

The cDNA coding for the CGL1 polypeptide has been identified by the inventors as being a nucleic acid having a complete identity with the nucleic acid referenced in the GenBanK data base under access number no AF052149, such a sequence being also referenced as the SEQ ID No 14 sequence of the sequence listing.

In the GenBank data base, the sequence NO AF052149 is simply identified as originating from a human messenger RNA randomly sequenced from a human mRNA bank. Reference AF052149 from the GenBanK does not contain any information about the existence of an open reading framework in the described sequence nor, all the more, the position of a possible open reading framework. Consequently, no information is given on a polypeptide which could be coded by such a sequence.

It has been shown according to the invention that the cDNA sequence coding for the CGL1 polypeptide, referenced as sequence SEQ ID No 14 of the sequence listing, has an open reading framework starting at the base A of the ATG codon, in position 345 and ending at the base A of the TGA codon in position 1541 of the SEQ ID No 14 sequence.

The open reading framework of the SEQ ID No 14 sequence codes for the CGL1 polypeptide with a SEQ ID No 1 amino acid sequence.

The invention consequently also relates to a nucleic acid coding for the CGL1 polypeptide characterized in that it comprises the SEQ ID No 14 nucleotide sequence.

Another object of the invention is also a nucleic acid coding for the CGL1 polypeptide characterized in that it comprises the polynucleotide ranging from the nucleotide in position 345 up to the nucleotide in position 1541 of the SEQ ID No 14 nucleotide sequence.

It also relates to any use of a nucleic acid coding for the CGL1 polypeptide, of a variant or of a fragment of such a nucleic acid for a diagnosis or a preventive or curative therapy for a pathology associated with a mutation or a defect in the expression of the gene coding for the CGL1 polypeptide, in particular a pathology listed in the [symbol] SUMMARY [symbol] section, including a lipodystrophia, preferably a congenital generalized lipodystrophia (CGL1), or a type 1 or type 2 diabetes.

Nucleic Acids Coding for a Variant of the CGL1 Polypeptide

As already set forth previously in the disclosure, the inventors have identified numerous mutations in the cgl1 gene in patients affected with congenital generalized lipodystrophia.

The coded mutated or truncated proteins coded by the cgl1 gene bearing various mutations are described hereunder, referring to the denomination of the various detected mutations.

F63 fsX75 Mutation

The cgl1 gene affected by such a mutation codes for the polypeptide with a SEQ ID No 2 sequence. The polypeptide of SEQ ID No 2 sequence has a truncated amino acid sequence compared to the normal CGL1 polypeptide having a SEQ ID No 1 sequence and additionally has the [symbol] RDCAFLLQDRL [symbol] C-terminal sequence which is not present in the normal CGL1 polypeptide, because of a change in the reading framework induced by the mutation.

F100fsX111 Mutation

The cgl1 gene bearing such a mutation codes for the polypeptide with a SEQ ID No 3 sequence. The polypeptide with a SEQ ID No 3 amino acid sequence is truncated compared to the normal CGL1 polypeptide and has the [symbol] KCMDSRIVLP [symbol] C-terminal sequence which is not present in the normal CGL1 polypeptide, because of a change in the reading framework induced by the mutation.

F105 fsX112 Mutation

The cgl1 gene bearing such a mutation codes for the polypeptide with a SEQ ID No 4 sequence. The polypeptide with a SEQ ID No 4 sequence is truncated compared to the normal CGL1 polypeptide and has the [symbol] SCYLRA [symbol] C-terminal sequence which is not present in the normal CGL1 peptide, because of a change in the reading framework induced by the mutation.

F105fs X111 Mutation

The cgl1 gene bearing such a mutation codes for the polypeptide with a SEQ ID No 5 sequence, which is truncated compared to the normal CGL1 polypeptide and has the [symbol] CYLRA [symbol] C-terminal sequence which is not present in the normal CGL1 polypeptide, because of a change in the reading framework induced by the mutation.

F108 fsX113 Mutation

The cgl1 gene bearing such a mutation codes for the polypeptide with a SEQ ID No 6 sequence, which is truncated compared to the normal CGL1 polypeptide and has a [symbol] NLRA [symbol] C-terminal sequence which is not present in the normal CGL1 polypeptide, because of a change in the reading framework induced by the mutation.

R138X Mutation

The cgl1 gene bearing such a mutation codes for the polypeptide with a SEQ ID No 7 sequence, having its amino acid sequence truncated compared to the normal CGL1 polypeptide.

[symbol]

Exon 4 Excision [symbol] Mutation

The cgl1 gene bearing such a mutation codes for a polypeptide comprising an amino acid deletion and codes for the polypeptide with a SEQ ID No 8 sequence. More specifically, it is a mutation of the first base of the alternative splicing site of the intron 4, inducing the excision of the exon 4. The excision of the exon 4 results in the deletion of 48 amino acids, while keeping the reading framework.

A 212P Mutation

The cgl1 gene bearing the A 212P mutation codes for the polypeptide with a SEQ ID No 9 sequence, resulting in a [symbol] false sense [symbol] mutation in the exon 6 inducing the substitution of the alanine residue in position 212 by a proline residue.

F 213fsX232 Mutation

The cgl1 gene bearing such a mutation codes for the polypeptide with a SEQ ID No 10 sequence, which is truncated compared to the normal CGL1 polypeptide and has a [symbol] TSASTRTSLGSDTCY TTSR [symbol] C-terminal sequence which is not present in the normal CGL1 polypeptide, because of a change in the reading framework.

The variants of the CGL1 polypeptide with sequences from SEQ ID No 2 to SEQ ID No 10 are within the scope of the invention.

Nucleic acids coding for the polypeptides with amino acid sequences from SEQ ID N⁰2 to SEQ ID No 10, as well as their complementary sequences, are also within the scope of the invention.

Table 2 hereafter sums up the various mutations of the cgl1 gene found by the inventors.

Fragments of the CGL1 Polypeptide

[symbol] Fragments [symbol] of the CGL1 polypeptide with a SEQ ID No 1 sequence are also within the scope of the invention. The fragments of the CGL1 polypeptide with a SEQ ID No 1 sequence preferably have a length of at least 10, 15, 20, 30, 40, 50, 75, 100, 150, 200, 2550, 300, 350 or 380 amino acids.

Fragments of the CGL1 polypeptide are useful especially for preparing antibodies specifically raised against such a protein.

The preferred fragments of the CGL1 polypeptide are the polypeptides respectively having the amino acid sequences SEQ ID No 11, SEQ ID No 12 and SEQ ID No 13.

A further object of the invention is a nucleic acid coding for a fragment of the CGL1 polypeptide such as defined herein above.

Nucleotide Probes and Primers

The invention also relates to the use of a nucleic acid coding for the CGL1 polypeptide or a fragment of the CGL1 polypeptide with a SEQ ID No 1 sequence for producing a specific nucleotide probe or primer for the cgl1 gene.

The invention also relates to the use of a nucleic acid coding for a variant of the CGL1 polypeptide for producing a specific nucleotide probe or primer for the mutated cgl1 gene.

Another object of the invention is also a hybridizing nucleotide probe or primer in highly stringent conditions defined in the present specification, specifically with the mutated cgl1 gene.

The nucleotide probes or primers according to the invention are preferably those characterized in that they specifically hybridize with a nucleic acid coding for one of the polypeptides with sequences SEQ ID No 2 to SEQ ID No 10, such polypeptides being coded by the mutated cgl1 gene.

Most preferably, the nucleotide probes and primers specifically hybridize with the cgl1 gene bearing a mutation selected amongst F63fsX75, F100fsX111, F105fsX112, F105fsX111, F108fsX113, R138X, A212P, F213fsX232, del/ins E5-6 et del E-6 mutations, represented in table 2.

Probes and primers hybridising with a non mutated nucleic acid coding for the CGL1 polypeptide, but which do not hybridize, in the specified hybridization conditions, with a nucleic acid bearing one of the above-mentioned mutations, are also within the scope of the invention.

The invention also relates to a nucleotide primer couple for detecting a mutation in the cgl1 gene, characterized in that it is the primer couple comprising the SEQ ID No 15 and SEQ ID No 16 nucleotide sequences defining the CA10 microsatellite marker.

A second primer couple according to the invention comprises the primers with sequences SEQ ID No 17 and SEQ ID No 18.

A nucleotide primer or probe according to the invention may be prepared by any adapted method well known to the man of the art, including through a cloning and restriction enzyme action or also through a direct chemical synthesis according to techniques such as the phosphodiester method from Narang et al. (1979) or from Brown et al. (1979), the diethylphosphoramidite method from Beaucage et al. (1980) or also the solid support technique disclosed in European Patent Application EP 0,707,592.

Each of the nucleic acids according to the invention, including the above-disclosed oligonucleotide probes and primers, may be labeled, if desired, through incorporating a marker able to be detected by spectroscopic, photochemical, biochemical, immunochemical as well as chemical means.

For example, such markers may consist in radioactive isotopes (32P, 33P, 3H, 35S), fluorescent molecules (5-bromodeoxyuridine, fluorescein, acetylaminofluorene, digoxigenin) as well as ligands such as biotin.

The probe labelling preferably occurs through incorporating labeled molecules within the polynucleotides through a primer extension, or also through an addition on the 5′ or 3′ ends.

Non radioactive labeling examples of nucleic acid fragments are more particularly disclosed in French Patent FR 78 109 75 or also in the articles by Urdea et al. (1988) or Sanchez-Pescador et al. (1988).

Advantageously, the probes according to the invention may show structural characteristics allowing for an amplification of the signal, such as the probes disclosed by Urdea et al. (1991) as well as in French Patent EP-0,225,807 (CHIRON).

The oligonucleotide probes according to the invention may be used for example in Southern type hybridizations with genomic DNA as well in corresponding messenger RNA hybridizations when the corresponding transcript expression is being sought in a sample.

The probes according to the invention may also be used for detecting PCR amplification products as well as for detecting mismatches.

Nucleotide probes or primers according to the invention may be immobilized on a solid substrate. Such solid substrates are well known to the man of the art and comprise microtitration plate well surfaces, polystyrene beds, magnetic beds, nitrocellulose strips, as well as microparticles like latex particles.

The present invention also relates to a method for detecting a mutation in a nucleic acid coding for the CGL1 polypeptide, characterized in that it comprises the steps of:
a) contacting one or more nucleotide probes such as defined herein above with the sample to be tested; and
b) detecting the complex optionally formed between the probe(s) and the nucleic acid present in the sample.

According to a particular embodiment of the detection method according to the invention, the oligonucleotide probe(s) is/are immobilized on a substrate.

According to another aspect, the oligonucleotide probes comprise a detectable marker.

The invention further relates to a set or kit for detecting a mutation in the cgl1 gene, characterized in that it comprises:
a) one or more nucleotide probes such as defined herein above; and
b) if need be, the necessary reactants for the hybridization reaction.

According to a first aspect, the detection set or kit is characterized in that the probe(s) is/are immobilised on a substrate.

According to a second aspect, the detection set or kit is characterized in that the oligonucleotide probes comprise a detectable marker.

According to a particular embodiment of the herein above described detecting kit, such a kit will comprise a plurality of oligonucleotide probes able to detect distinct mutations in the cgl1 gene.

The probes according to the invention being immobilized on a substrate may be organized in matrices such as [symbol] DNA chips [symbol] . Such organized matrices have been particularly disclosed in U.S. Pat. No. 5,143,854 and PCT Applications WO 90/15070 and 92/192.

Substrate matrices on which the oligonucleotide probes have been immobilized at a high density are, for example, disclosed in U.S. Pat. No. 5,412,087 and in PCT Application WO 95/11995.

The nucleotide primers according to the invention are preferably used to detect a mutation in a nucleic acid coding for the CGL1 polypeptide, and consequently selectively amplify all or part of a nucleic acid bearing a mutation of the cgl1 gene.

A further object of the invention relates to a method for detecting a mutation in a nucleic acid coding for the CGL1 polypeptide, characterized in that it comprises the steps of:
a) contacting the sample to be tested with one or more nucleotide primers such as defined herein above; and
b) detecting the amplified nucleic acids.

Another further object of the invention is to provide a set or kit for detecting a mutation in a nucleic acid coding for the CGL1 polypeptide, characterized in that it comprises:
a) one or more nucleotide primers such as defined herein above; and
b) if need be, the necessary reactants for the amplification reaction.

The nucleotide primers according to the invention are particularly useful within the scope of the method for genotyping subjects and/or genotyping populations, including within the scope of association studies between the particular allelic forms or forms of particular allele groups (haplotypes) in CGL affected subjects and the existence of a particular phenotype (character) in such subjects, for example a predisposition of those subjects for developing pathologies linked to a generalized dystrophia, a diabetes or obesity.

The nucleotide probes and primers according to the invention are also useful as detection means for the purpose of a diagnosis for CGL1 linked pathologies, more particularly the pathologies as listed in the [symbol] SUMMARY [symbol] section of the present disclosure, including lipoatrophic diabetes, in particular within the scope of a prenatal diagnosis.

Recombinant Vectors

The invention would also relate to a recombinant vector comprising a nucleic acid coding for the CGL1 polypeptide with a SEQ ID No 1 amino acid sequence or a fragment or a variant of the CGL1 polypeptide.

Advantageously, a recombinant vector comprising a nucleic acid coding for a variant of the CGL1 polypeptide will comprise a nucleic acid coding for a polypeptide selected amongst polypeptides with an amino acid sequence from SEQ ID No 2 to SEQ ID No 10.

Advantageously, a recombinant vector comprising a nucleic acid coding for a fragment of the CGL1 polypeptide will comprise a nucleic acid coding for a polypeptide selected amongst polypeptides with an amino acid sequence from SEQ ID No 11 to SEQ ID No 13.

According to a first preferred embodiment, the nucleic acid coding for the CGL1 polypeptide comprises the cgl1 gene contained in the BAC vector referred to as RP11-831H9, partial nucleotide sequences of which are referenced in the GenBank data base under access number No AP001458.

According to a second preferred embodiment, the nucleic acid coding for the CGL1 polypeptide is a nucleic acid comprising the SEQ ID No 14 nucleotide sequence.

According to a third preferred embodiment, the nucleic acid coding for the CGL1 polypeptide comprises the polynucleotide ranging from the nucleotide in position 345 up to the nucleotide in position 1541 in the SEQ ID No 14 nucleotide sequence.

The term “vector” as used herein refers to a circular or linear DNA or RNA molecule indiscriminately under the form of a single strand or a double strand.

According to a first embodiment, a recombinant vector according to the invention is used in order to amplify the nucleic acid being inserted after a transformation or transfection of the desired cell host.

According to a second embodiment, there are provided expression vectors comprising, in addition to a nucleic acid in accordance with the invention, regulatory sequences allowing for directing the transcription and/or the translation thereof.

According to an advantageous embodiment, a recombinant vector according to the invention will for example comprise the following elements:
(1) regulatory elements for the expression of the nucleic acid to be inserted, such as promoters and enhancing sequences (“enhancers”);
(2) the coding sequence included in the nucleic acid in accordance with the invention to be inserted in such a vector, said coding sequence being put in phase with the regulatory signals as described in (1); and
(3) appropriate transcription initiation and stoppage sequences.

Additionally, the recombinant vectors according to the invention would be likely to include one or more replication origins amongst the cell hosts wherein their amplification or their expression is being sought, markers or selection markers.

By way of examples, the bacterial promoters could be LacI, LacZ promoters, promoters of the polymerase RNA of the T3 or T7 bacteriophage, PR or PL promoters of the lambda phage.

The promoters for eukaryotic cells would comprise the thymidine kinase promoter of the HSV virus as well as the mouse's metallothionein-L promoter.

Generally, in selecting an adapted promoter, the man of the art could advantageously refer to the above-mentioned work by Sambrook et al. (1989) or also to the techniques as described by Fuller et al. (1996).

When the expression of the genomic sequence of the cgl1 gene is desired, usage will be preferably made of vectors able to include large insertion sequences. In such a particular embodiment, one will preferably use bacteriophage vectors, such as P1 bacteriophage vectorS such as the p158 vector or also the p158/neo8 vector described by Sternberg (1992, 1994).

The preferred bacterial vectors according to the invention are for example the pBR322(ATCC37017) vectors as well as vectors such as pAA223-3 (Pharmacia, Uppsala, Sweden), and pGEM1 (Promega Biotech, Madison, Wis., USA).

Other commercialized vectors may also include vectors such as pQE70, pQE60, pQE9 (Qiagen), psiX174, pBluescript SA, pNH8A, pNH16A, pNH18A, pNH46A, pWLNEO, pSV2CAT, pOG44, pXT1, pSG(Stratagene).

They may also be vectors of the baculovirus type such as the pVL1392/1393 vector (Pharmingen) used for transfecting cells from the Sf9 lineage (ATCC NoCRL 1711) derived from Spodoptera frugiperda.

They may also be adenoviral vectors such as type 2 or 5 human adenovirus.

A recombinant vector according to the invention may also be a retroviral vector or also an adeno-associated vector (AAV). Such adeno-associated vectors are for example disclosed by Flotte et al. (1992), Samulski et al. (1989), as well as McLaughlin BA et al. (1996).

In order to allow for the expression of the polynucleotides according to the invention, the latter should be introduced into a host cell. Introducing polynucleotides according to the invention into a host cell could occur in vitro, using techniques well known to the man of the art for transforming or transfecting cells, either in a primary culture, or under the form of cell lineages. The in vivo introduction of polynucleotides according to the invention may also be performed for preventing or treating pathologies associated with lipodystrophia, diabetes or obesity, such as the pathologies listed in the [symbol] SUMMARY [symbol] section of the present disclosure.

In order to introduce the polynucleotides or the vectors into a host cell, the man of the art could advantageously refer to various techniques, such as the calcium phosphate precipitation technique (Graham et al., 1973; Chen et al., 1987), le DEAE Dextran (Gopal, 1985), the electroporation (Tur-Kaspa, 1896; Potter et al., 1984), the direct microinjection (Harland et al., 1985), the DNA-charged liposomes (Nicolau et al., 1982, Fraley et al., 1979).

Useful Vectors in the Implementation of Genic Therapy Methods

Once the polynucleotide has been introduced into the host cell, it can be stably integrated within the cell genome. The integration can occur in a precise site in the genome, through an homologous recombination, or could be integrated randomly. In some embodiments, the polynucleotide can be steadily maintained in the host cell under the form of an episome fragment the episome comprising sequences allowing for the preservation and the replication of the latter, either independently or in a synchronised way with the cell cycle.

According to a particular embodiment, a method for introducing a polynucleotide according to the invention into a host cell, in particular a host cell originating from a mammal, in vivo, comprises a step wherein a preparation is introduced comprising a pharmaceutically compatible vector and a “bare” polynucleotide according to the invention, placed under the control of appropriate regulatory sequences, through a local injection at the level of the selected tissue, for example a smooth muscle tissue, the “bare” polynucleotide being absorbed by the cells of such a tissue.

Compositions for use in vitro and in vivo comprising “bare” polynucleotides are for example disclosed in PCT Application WO 95/11307 (Institut Pasteur, Inserm, Ottawa University) as well as in the articles by Tacson et al. (1996) and Huygen et al. (1996).

According to a specific embodiment of the invention, a composition is provided for producing a CGL1 polypeptide in vivo. Such a composition comprises a polynucleotide coding for the CGL1 polypeptide placed under the control of appropriate regulatory sequences, in a solution in a physiologically acceptable vector.

The vector amount being injected to the selected host organism is varied according to the injection site. Indicatively, from about 0.1 to about 100 μg of the polynucleotide coding for the CGL1 polypeptide can be injected into the body of an animal, preferably a patient subject to developing a lipodystrophia, a diabetes or obesity, in particular a CGL syndrome, or having already developed such a disease.

In addition, another object of the invention is the use of a nucleic acid coding for the CGL1 polypeptide or the use of a recombinant vector comprising such a nucleic acid for producing a drug for preventing or treating a lipodystrophia, and preferably a congenital generalized lipodystrophia (CGL), or also the Lawrence syndrome or obesity.

The invention also relates to the use of a nucleic acid coding for the CGL1 polypeptide or the use of a recombinant vector comprising such a nucleic acid for producing a drug for preventing or treating diabetes, obesity or an insulin-resistance syndrome.

The invention also relates to a pharmaceutical composition for preventing or treating a lipodystrophia, preferably a congenital generalized lipodystrophia (CGL), or also the Lawrence syndrome or obesity, comprising a nucleic acid coding for the CGL1 polypeptide, or comprising a recombinant vector comprising such a nucleic acid, in association with one or more physiologically compatible excipients.

The invention also relates to a pharmaceutical composition for preventing or treating diabetes or an insulin-resistance syndrome, comprising a nucleic acid coding for the CGL1 polypeptide, or comprising a recombinant vector comprising such a nucleic acid, in association with one or more physiologically compatible excipients.

Vectors Useful in Somatic Genic Therapy Methods and Compositions Containing Such Vectors

The present invention also relates to a new therapeutic approach for treating the pathologies listed in the [symbol] SUMMARY [symbol] section, in particular pathologies associated to lipodystrophia, diabetes or obesity. It proposes an advantageous solution to the prior art inconveniences, showing the possibility to treat pathologies associated to lipodystrophia or diabetes, in particular congenital generalized lipodystrophia (CGL) through a genic therapy, through the transfer and the in vivo expression of a gene coding for the CGL1 polypeptide.

The genic therapy consists in correcting a deficiency or an anomaly (mutation, aberrant expression, etc.) or providing the expression of a protein of therapeutic interest through the introduction of genetic information into the affected cell or organ. Such genetic information could be introduced either ex vivo into a cell extracted from the organ, the modified cell being then reintroduced into the organism, or directly in vivo into the appropriate tissue. In this second case, various techniques exist, amongst which various transfecting techniques involving complexes of DNA and DEAE-Dextran (Pagano et al., J. Virol. 1 (1967) 891), DNA and nuclear proteins (Kaneda et al., Science 243 (1989) 375), DNA and lipids (Felgner et al., PNAS 84 (1987) 7413), the use of liposomes (Fraley et al., J. Biol. Chem. 255 (1980) 10431), etc. More recently, the use of viruses as vectors for transferring genes has been found as a promising alternative to such physical transfection techniques. In this respect, various viruses have been tested for their ability to infect some cell populations, in particular, retroviruses (RSV, HMS, MMS, etc.), the HSV virus, the adeno-associated viruses, and the adenoviruses.

The present invention relates thus also to a new therapeutic approach for treating pathologies associated with a mutation or a deficiency in the expression of the CGL1 gene, preferably pathologies listed in the [symbol] SUMMARY [symbol] section of the present disclosure, more particularly, pathologies associated with the CGL, consisting in transferring and expressing in vivo genes coding for CGL1.

The present invention also results from demonstrating that adenoviruses are particularly efficient vectors for transferring and expressing a nucleic acid coding for the CGL1 polypeptide. In particular, the present invention shows that using recombinant adenoviruses as vectors makes it possible to obtain sufficiently high expression levels of such a gene in order to produce the desired therapeutic effect. Other viral vectors such as retroviruses or adeno-associated viruses (AAV) allowing for a stable expression of the gene are also objects of the invention.

The present invention thus provides a new approach for treating and preventing in particular pathologies associated with a lipodystrophia or diabetes.

Another object of the invention is a defective recombinant virus comprising a nucleic acid coding for the CGL1 polypeptide involved in the CGL syndrome.

The invention also relates to the use of such a defective recombinant virus for preparing a pharmaceutical composition for treating and/or preventing diseases having a lipodystrophia, a diabetes, the Lawrence syndrome, obesity, the insulin-resistance syndrome.

The present invention also relates to the use of such ex vivo genetically modified cells by a virus such as described herein above, or of cells producing such viruses, implanted into the organism, allowing for an in vivo extended and efficient expression of the CGL1 polypeptide.

According to the invention, one can incorporate a sequence of DNA coding for the CGL1 polypeptide in a viral vector, and such vector allows to efficiently express a mature biologically active form. More particularly, the invention shows that the in vivo expression of the CGL1 polypeptide could be obtained through direct administration of an adenovirus or through implantation of a producing cell or a cell genetically modified by an adenovirus or by a retrovirus incorporating such a DNA.

The present invention is particularly advantageous as it makes it possible to induce a controlled expression and without any deleterious effect of CGL1 in organs which are not normally involved with the expression of such a protein. In particular, a significant release of CGL1 polypeptide is obtained through the implantation of producing cells of vectors of the invention, or ex vivo infected by vectors of the invention.

The nucleic acid coding for the CGL1 polypeptide could be a cDNA, a genomic DNA (gDNA), a RNA (in the case of retroviruses) or a hybrid construct comprising for example a cDNA where one or more introns would be inserted. It could also be synthetic or semi-synthetic sequences. In a particularly advantageous way, a cDNA or a gDNA is used. In particular, using a gDNA allows for a better expression in the human cells. In order to allow their incorporation into a viral vector according to the invention, such sequences are advantageously modified, for example through directed mutagenesis, in particular for inserting appropriate restriction sites. The sequences described in the prior art are indeed not built up for a use according to the invention, and preliminary adaptations could prove to be necessary in order to obtain important expressions. Within the scope of the present invention, a nucleic sequence coding for a human CGL1 polypeptide is preferably used.

According to other embodiments, the nucleic acid coding for a polypeptide coded by a gene orthologous to the cgl1 gene could originate from another mammal, for example from mouse. In this case, nucleic acids such as described in the GenBank data base under access numbers AF069954, AB041588 and AB030196 or the genomic sequence will be preferably used.

On the other hand, it is also possible to use a construct coding for a CGL1 polypeptide derivate. A CGL1 polypeptide derivate comprises for example any sequence obtained through mutation, deletion and/or addition compared to the native sequence, and coding for a product maintaining the preventive or curative activity of the CGL. Such modifications could be achieved using techniques known to the man of the art (see general techniques of molecular biology hereinafter).

Such derivates are in particular molecules with a higher affinity for their fixing sites, molecules having higher protease resistance, molecules having a higher therapeutic efficiency or fewer side effects, or optionally new biological properties. The derivates also include modified DNA sequences allowing for an in vivo improved expression.

In a first embodiment, the present invention relates to a defective recombinant virus comprising one cDNA sequence coding for the CGL1 polypeptide.

In another preferred embodiment of the invention, the DNA sequence is a gDNA sequence.

The vectors of the invention could be prepared from different virus types. Preferably, vectors derived from adenoviruses, adeno-associated (AAV) viruses, herpes viruses (HSV) or retroviruses will be used. It is most particularly advantageous to use an adenovirus, for a direct administration or for the ex vivo modification of cells intended to be implanted, or a retrovirus, for the implantation of producing cells.

The viruses according to the invention are defective, i.e. they are unable to autonomously replicate in the target cell. Generally, the genome of the defective viruses used within the scope of the present invention is therefore deprived of at least the sequences required for replicating said virus in the infected cell. Such regions could either be eliminated (totally or partially), or be made non-functional, or substituted by other sequences and especially by the nucleic sequence coding for the CGL1 polypeptide. Preferably, the defective virus however maintains the sequences of its genome being required for the encapsidation of the viral particles.

As this more particularly relates to adenoviruses, various serotypes, the structure and properties of which somewhat vary, have been characterized. Amongst such serotypes, type 2 or 5 human adenoviruses (Ad 2 or Ad 5) or adenoviruses from animal origin (see Application WO 94/26914) are preferably used within the scope of the present invention. Amongst the adenoviruses from animal origin useful within the scope of the present invention one can mention adenoviruses from canine, bovine, murine, (example: Mav1, Beard et al., Virology 75 (1990) 81), ovine, porcine, avian as well as simian origin (example: SAV). Preferably, the adenovirus from animal origin is a canine adenovirus, more preferably a CAV2 adenovirus [Manhattan or A26/61 (ATCC VR-800) strain for example]. Preferably, within the scope of the invention adenoviruses from human or canine origin or mixed origin will be used. Preferably, the defective adenoviruses of the invention comprise ITR, a sequence allowing for the encapsidation and the sequence coding for the CGL1 polypeptide. Advantageously, in the genome of the adenoviruses of the invention, the E1 region at least is made non functional. Most preferably, in the genome of the adenoviruses of the invention, the E1 gene and at least one of the E2, E4, L1-L5 genes are non functional. The subject viral gene could be made non functional by any technique known to the man of the art, and more particularly through total suppression, substitution, partial deletion, or addition of one or more bases in the subject gene(s). Such modifications could be achieved in vitro (on isolated DNA) or in situ, for example, using genetic engineering techniques, as well as treating using mutagenic agents. Other regions could also be modified, and more particularly the E3 region (WO95/02697), the E2 region (WO94/28938), the E4 region (WO94/28152, WO94/12649, WO95/02697) and the L5 region (WO95/02697). According to a preferred embodiment, the adenovirus according to the invention comprises a deletion in the E1 and E4 regions and the sequence coding for the CGL1 polypeptide is inserted at the level of the inactivated E1 region. According to another preferred embodiment, it comprises a deletion in the E1 region at which level are inserted the E4 region and the sequence coding for the CGL1 polypeptide (French Patent Application FR94 13355).

The defective recombinant adenoviruses according to the invention could be prepared using any technique known to the man of the art (Levrero et al., Gene 101 (1991) 195, EP 185 573; Graham, EMBO J. 3 (1984) 2917). In particular, they may be prepared through homologous recombination between an adenovirus and a plasmid bearing a nucleic acid coding for the CGL1 polypeptide. The homologous recombination occurs after co-transfection of said adenovirus and plasmid in an appropriate cell lineage. The cell lineage to be used should preferably (i) either be able to be transformed by said elements, and (ii), comprise sequences able to complement part of the defective adenovirus genome, preferably under an integrated form so as to avoid the recombination risks. As an example of lineage, one could mention the 293 human kidney embryo lineage (Graham et al., J. Gen. Virol. 36 (1977) 59) which more particularly contains, being integrated in its genome, the left part of the genome of a Ad5 adenovirus (12%) or of lineages able to complement the E1 and E4 functions such as disclosed, more specially, in Applications WO 94/26914 and WO95/02697.

Thereafter, the adenoviruses having been multiplied are recovered and purified according to the conventional molecular biology techniques, as illustrated in the examples.

As to the adeno-associated viruses (AAV), they are viruses with a relatively reduced size DNA, integrating into the genome of the cells they infect, in a stable and site-specific way. They are able to infect a broad spectrum of cells, without inducing any effect on the cell growth, morphology or differentiation. Additionally, they do not seem to be involved in human pathologies. The AAV genome has been cloned, sequenced and characterized. It comprises approximately 4,700 bases, and contains at each end an inverted repeated region (ITR) comprising about 145 bases, acting as the replication origin for the virus. The remainder of the genome is divided into 2 essential regions carrying the encapsidation functions: the left part of the genome, containing the rep gene involved in the viral replication and the viral gene expression; the right part of the genome, containing the cap gene coding for the virus capsid proteins.

The use of AAV derived vectors for in vitro and in vivo transferring genes has been described in the literature (see in particular WO 91/18088; WO 93/09239; U.S. Pat. No. 4,797,368, U.S. Pat. No. 5,139,941, EP 488 528). Such applications disclose various AAV derived constructs, wherein the rep and/or cap genes are deleted and replaced by a gene of interest, and their use for in vitro transferring (on cultured cells) or in vivo transferring (directly into an organism) said gene of interest. However, none of those documents discloses or suggests the use of a recombinant AAV for in vivo or ex vivo transferring or expressing the CGL1 polypeptide, nor the advantages of such a transfer. The defective recombinant AAVs according to the invention could be prepared through co-transfection, in a cell lineage infected by a human auxiliary virus (for example an adenovirus), of a plasmid containing the sequence coding for the CGL1 polypeptide bordered with the AAV reversed repeated regions (ITR), and of a plasmid carrying the AAV encapsidation genes (rep and cap genes). The resulting recombinant AAVs are then purified by conventional techniques.

As to the herpes viruses and retroviruses, the recombinant vector construction has been widely described in the literature: see more particularly Breakfield et al., New Biologist 3 (1991) 203; EP 453242, EP178220, Bernstein et al. Genet. Eng. 7 (1985) 235; McCormick, BioTechnology 3 (1985) 689, etc.

In particular, retroviruses are integrative viruses, infecting dividing cells. The retrovirus genome essentially comprises two LTRs, an encapsidation sequence and three coding regions (gag, pol and env). In the recombinant vectors derived from retroviruses, the gag, pol and env genes are generally deleted, totally or partially, and replaced by a sequence of heterologous nucleic acid of interest. Such vectors could be obtained from various retrovirus types such as for example MoMuLV (“murine moloney leukemia virus”; also referred to as MoMLV), MSV (“murine moloney sarcoma virus”), HaSV (“harvey sarcoma virus”); SNV (“spleen necrosis virus”); RSV (“rous sarcoma virus”) as well as Friend's virus.

To construct the recombinant retroviruses comprising a sequence coding for the CGL1 polypeptide according to the invention, a plasmid containing in particular LTRs, the encapsidation sequence and said coding sequence coding is generally constructed, and then used for transfecting a so-called encapsidation cell lineage, able to bring in trans position the deficient retroviral functions in the plasmid. Generally, the encapsidation lineages are therefore able to express the gag, pol and env genes. Such encapsidation lineages have been disclosed in the prior art, and more particularly the PA317 lineage (U.S. Pat. No. 4,861,719); the PsiCRIP lineage (WO90/02806) and the GP+envAm-12 lineage (WO89/07150). On the other hand, the recombinant retroviruses may contain modifications at the LTR level for suppressing the transcriptional activity as well as extended encapsidation sequences, comprising part of the gag gene (Bender et al., J. Virol. 61 (1987) 1639). The resulting recombinant retroviruses are then purified by conventional techniques.

For implementing the present invention, it is mostly advantageous to use a defective recombinant adenovirus. The results given hereinafter do show the particularly interesting properties of the adenoviruses for in vivo expressing a protein with a cholesterol carrying activity. The adenoviral vectors according to the invention are particularly advantageous for a direct in vivo administration of a purified suspension, or for the ex vivo transformation ex vivo of, in particular autologous cells, with a view to their implantation. Moreover, the adenoviral vectors according to the invention additionally offer important advantages, such as their very high infection efficiency, allowing for infections to be achieved from low viral suspension volumes.

According to another particularly advantageous embodiment of the invention, a lineage producing retroviral vectors containing the sequence coding for the CGL1 polypeptide is used, for an in vivo implantation. The lineages useful to this end are more particularly the PA317 (U.S. Pat. No. 4,861,719), PsiCrip (WO90/02806) and GP+envAm-12 (U.S. Pat. No. 5,278,056) cells, modified so as to allow for the production of a retrovirus containing a nucleic sequence coding for the CGL1 polypeptide according to the invention. For example totipotent strain cells, precursors of the blood cell lineages, could be taken and isolated from the subject. Such cultured cells could then be transfected by the retroviral vector containing the sequence coding for the CGL1 polypeptide under the control of viral, non viral, non viral and specific promoters for the macrophages or also under the control of its own promoter.

Advantageously, in the vectors according to the invention, the sequence coding for the CGL1 polypeptide is placed under the control of signals allowing for its expression in the infected cells. These can be homologous or heterologous expression signals, i.e. signals differing from those naturally responsible for the expression of the CGL1 polypeptide. These can more particularly be sequences responsible for the expression of other proteins, or synthetic sequences. More specifically, these could be sequences of eukaryotic or viral genes or derived sequences, stimulating or repressing the transcription of a gene in a specific or non specific way and in an inducible or non indicible way. By way of an example, these could be promoter sequences originating from the genome of the cell to be infected, or the genome of a virus, and especially, the promoters of the E1A, MLP adenovirus genes, the CMV, LTR-RSV promoter, etc. Amongst the eukaryotic promoters, one can also mention ubiquitous promoters (HPRT, vimentin, α-actin, tubulin, etc.), promoters of intermediate filaments (desmin, neurofilaments, keratin, GFAP, etc), promoters of therapeutic genes (types MDR, CFTR, VIII factor, etc), tissue specific promoters (pyruvate kinase, villin, promoter of the intestinal linking protein for fatty acids, promoter of the α-actin of smooth muscle cells, liver specific promoters; Apo AI, Apo AII, human albumin, etc.) as well as promoters responding to a stimulus (steroid hormone receptor, retinoic acid receptor, etc.). In addition, such expression sequences could be modified through the addition of activation, regulating, etc. sequences. Moreover, when the inserted gene does not comprise any expression sequences, it could be inserted into the defective virus genome downstream such a sequence.

In a particular embodiment, the invention relates to a defective recombinant virus comprising a nucleic sequence coding for the CGL1 polypeptide under the control of a promoter selected amongst LTR-RSV or the early CMV promoter.

As indicated herein above, the present invention also relates to any use of a virus such as descried hereinabove for preparing a pharmaceutical composition for treating and/or preventing pathologies showing a lipodystrophia or a diabetes.

The present invention also relates to a pharmaceutical composition comprising one or more defective recombinant viruses, such as previously described. Such pharmaceutical compositions can be formulated with a view to topic, oral, parenteral, intranasal, intraveinous, intramuscular, subcutaneous, intra-ocular, transdermal, etc. administration. Preferably, the pharmaceutical compositions of the invention contain a pharmaceutically acceptable carrier for an injectable formulation, more particularly for an intraveinous injection, such as for example in the patient's portal vein.

These can be more particularly sterile, isotonic solutions or dry compositions, including freeze-dried ones, which, through the addition, depending on the case, of sterilised water or a saline solution, allow for the formation of injectable solutes. The direct injection into the patient's portal vein is advantageous as it allows to target the infection at the liver level and thereby to concentrate the therapeutic effect at the level of that organ.

The doses of defective recombinant virus to be used for the injection can be adapted depending on various parameters, particularly, depending on the viral vector, the administration mode being used, the subject pathology as well as the desired treatment duration. Generally, the recombinant adenoviruses according to the invention are formulated and administered in the form of doses ranging from 10⁴ to 10¹⁴ pfu/ml, preferably from 10⁶ to 10¹⁰ pfu/ml. The term pfu (“plaque forming unit”) corresponds to the infectious power of a virus solution, and is determined by infecting an appropriate cell culture and measuring, generally after 48 hours, the number of infected cell ranges. The techniques for determining the pfu titre of a viral solution are well documented in the literature.

Regarding the retroviruses, the compositions according to the invention can directly comprise the producing cells, with a view to their implantation.

With this respect, another object of the invention relates to any mammalian cell infected by one or more defective recombinant viruses such as described hereinabove. More particularly, the invention relates to any human cell population infected by such viruses. These can be in particular cells from blood origin (totipotent strain cells or precursors), fibroblasts, myoblasts, hepatocytes, keratinocytes, smooth and endothelial muscle cells, glial cells, etc.

The cells according to the invention can originate from primary cultures. Those can be taken out using any technique known to the man of the art, and then cultured in conditions allowing for their proliferation. As far as more particularly fibroblasts are concerned, these can be easily obtained from biopsies, for example according to the technique described by Ham [Methods Cell. Biol., 21a (1980) 255]. Such cells can be used directly for infection by the viruses, or stored, for example, using deep freezing, for creating autologous banks, with a view to a subsequent use. The cells according to the invention can also be secondary cultures, obtained for example from pre-established banks (see for example EP 228458, EP 289034, EP 400047, EP 456640).

The cultured cells are then infected by the recombinant viruses for providing them with the ability to produce the CGL1 polypeptide. The infection is achieved in vitro using techniques known to the man of the art. In particular, depending on the cell type being used and the desired number of virus copies per cell, the man of the art can adapt the infection multiplicity and optionally the achieved number of infection cycles. It should be understood that such steps will be conducted in appropriate sterility conditions when the cells are intended for an in vivo administration. The doses of recombinant viruses to be used for infecting the cells can be adapted by the man of the art depending on the aim being sought. The conditions as described hereinabove for the in vivo administration can be applied to the in vitro infection. For the infection by retroviruses, it also possible to co-culture the cells to be infected with recombinant retroviruses producing cells according to the invention. This allows for the retrovirus purification to be omitted.

Another object of the invention relates to an implant comprising mammalian cells infected by one or more defective recombinant viruses such as described hereinabove or recombinant virus producing cells, and an extracellular matrix. Preferably, the implants according to the invention comprise 10⁵ to 10¹⁰ cells. More preferably, they comprise 10⁶ to 10⁸ cells.

More particularly in the implants of the invention, the extracellular matrix comprises a gelling compound and optionally a substrate allowing the cells anchorage.

For preparing implants according to the invention, various types of gelling compounds can be used. The gelling compounds are used for including cells into a matrix having the structure of a gel, and for enhancing the anchorage of the cells on the substrate, if need be. Various cell adhering agents could therefore be used like gelling compounds, including collagen, gelatine, glycosaminoglycanes, fibronectin, lectins, etc. Preferably, collagen is used within the scope of the present invention. This can be collagen from human, bovine or murine origin. More preferably, type I collagen is used.

As indicated hereinabove, the compositions according to the invention advantageously comprise a substrate allowing for the anchorage of the cells. The term anchorage refers to any biological and/or chemical and/or physical interaction form leading to the adhesion and/or the attachment of the cells on the substrate. On the other hand, the cells may either overlap the substrate being used, or enter such a substrate, or both. A solid, non toxic and/or biocompatible substrate is preferably used within the scope of the invention. More particularly, polytetrafluoroethylene (PTFE) fibres or a biological origin substrate can be used.

The present invention thus provides for a very efficient means for treating or preventing CGL associated pathologies, such as lipodystrophia or a diabetes.

In addition, such a treatment can also relates to man as well as any animal such as ovine and bovine races, pet animals (dogs, cats, etc), horses, fishes, etc.

Recombinant Host Cells

The invention also relates to a recombinant host cell characterized in that it is transfected or transformed by a nucleic acid coding for the CGL1 polypeptide or a fragment of the CGL1 polypeptide, as well as by a recombinant vector comprising a nucleic acid coding for the CGL1 polypeptide or a fragment of the CGL1 polypeptide.

The preferred host cells according to the invention are for example as follows:
a) prokaryotic host cells: Escherichia coli (DH5-α strain), Bacillus subtilis and Salmonella typhimurium strains as well as species strains such as Pseudomonas, Streptomyces et Staphylococcus;
b) eukaryotic host cells: HeLa cells (ATCC NoCCL2), Cv 1 cells (ATCC NoCCL70), COS cells (ATCC NoCRL 1650), Sf-9 cells (ATCC NoCRL 1711), CHO cells (ATCC NoCCL-61) as well as 3T3 cells (ATCC NoCRL-6361).

According to the invention, hypophysial cell, CNS cells, such as neuronal cells, as well as pre-adipocytic cells.

Another object of the invention is also the use of a recombinant host cell such as defined hereinabove for producing a drug for preventing or treating a lipodystrophia, preferably a congenital generalized lipodystrophia (CGL).

The invention also relates to the use of a recombinant host cell such as defined hereinabove for producing a drug for preventing or treating a diabetes.

Still another object of the invention is to provide a pharmaceutical composition designed for preventing or treating a lipodystrophia, preferably, a congenital generalized lipodystrophia (CGL) comprising a recombinant host cell such as defined hereinabove, in association with one or more physiologically compatible excipients.

It also relates to a pharmaceutical composition for preventing or treating a diabetes comprising a recombinant host cell such as defined hereinabove, in association with one or more physiologically compatible excipients.

Polypeptides According to the Invention

Another object of the invention is to provide the SEQ ID No 1 sequence CGL1 polypeptide.

It also relates to a fragment of the CGL1 polypeptide such as previously described in the disclosure.

It also relates to a variant of the CGL1 polypeptide such as previously described in the present disclosure.

The preferred variants of the CGL1 polypeptide according to the invention are polypeptides with amino acid sequences from SEQ ID No 2 to SEQ ID No 10.

Another object of the invention is also to provide a polypeptide having at least 90%, advantageously at least 91%, 92%, 93%, 94%, 95%, 96%, 97%, 98%, 99% or at least 99.5% amino acid identity with the CGL1 polypeptide as well as with a fragment or a variant of the CGL1 polypeptide.

The preferred fragments of the CGL1 polypeptide are polypeptides with amino acid sequences from SEQ ID No 11 to SEQ ID No 13.

The invention also relates to the use of the CGL1 polypeptide for producing a drug for preventing or treating a lipodystrophia, preferably a congenital generalized lipodystrophia (CGL).

It also relates to use of the CGL1 polypeptide for producing a drug for preventing or treating a diabetes.

The invention also relates to a pharmaceutical composition for preventing or treating a pathology linked to a mutation or an expression default of the gene coding for the CGL1 polypeptide, preferably a pathology listed in the [symbol] SUMMARY [symbol] section of the present disclosure, including a lipodystrophia, preferably a congenital generalized lipodystrophia (CGL), the Lawrence syndrome or obesity comprising a CGL1 polypeptide in association with one or more physiologically compatible excipients.

The invention also relates to a pharmaceutical composition for preventing or treating a diabetes or for treating a diabetes or the insulin-resistance syndrome comprising a CGL1 polypeptide in association with one or more physiologically compatible excipients.

Generally, the polypeptides according to the present invention are in an isolated or purified form.

Generally, the nucleic acids according to the present invention are in an isolated or purified form.

The above-mentioned pharmaceutical compositions comprise a therapeutically efficient amount of the CGL1 polypeptide, in particular of the SEQ ID No 1 sequence CGL1 polypeptide.

Such pharmaceutical compositions are advantageously adapted for, for example, a parenteral administration of an amount of CGL1 polypeptide ranging from 1 μg/kg/day to 10 mg/kg/day, preferably at least 0.01 mg/kg/day and most preferably from between 0.01 and 1 mg/kg/day.

The invention also relates to a method for producing a CGL1 polypeptide or a fragment of the CGL1 polypeptide as well as a variant of the latter, said method comprising the following steps of:
a) inserting a nucleic acid coding for said polypeptide in an appropriate vector;
b) culturing, in an appropriate culture medium, a host cell preliminarily transformed or transfected with the recombinant vector in step a);
c) recovering the conditioned culture medium or lysing the host cell, for example through sonication or through osmotic shock;
d) separating and purifying said polypeptide from said culture medium or also from the cell lysates obtained in step c);
e) if need be, characterizing the resulting recombinant polypeptide.

The peptides according to the invention may be characterized by fixation on an immunoaffinity chromatographic column on which the antibodies raised against such a polypeptide or against a fragment or a variant thereof have been preliminarily immobilized.

According to another aspect, a recombinant polypeptide according to the invention can be purified by passing through an appropriate set of chromatographic columns according to the methods known to the man of the art and described, for example, in F. Ausubel et al. (1989).

A polypeptide according to the invention can also be prepared using conventional chemical synthesis techniques indiscriminately in a homogeneous solution or in solid phase.

By way of an illustration, a polypeptide according to the invention could be prepared by the technique or in a homogeneous solution described by Houben Weyl (1974) or also by the solid phase synthesis technique described by Merrifield (1965a; 1965b).

Are also within the scope of the inventions the so-called “homologous” polypeptides to any of the polypeptides with amino acid sequences SEQ ID NO 1 to 13, or to their fragments or variants.

Such homologous polypeptides have amino acid sequences having one or more substitutions of an amino acid by an equivalent amino acid, compared to the reference polypeptides.

The expression equivalent amino acid according to the present invention refers, for example, to the substitution of a residue in the L form by a residue in the D form or also the substitution of a glutamic acid (E) by a pyroglutamic acid according to techniques well known to the man of the art. By way of illustration, the synthesis of a peptide containing at least one residue in the D form is described by Koch (1977).

According to another aspect, are also considered as equivalent amino acids, two amino acids belonging to the same class, i.e. two acidic, basic, non polar or even non charged polar amino acids.

The scope of the invention also covers polypeptides comprising at lest one non peptide link such as a retro-inverso link (NHCO), a carba link (CH₂CH₂) or also a ketomethylene link (CO—CH₂).

Preferably, the polypeptides according to the invention comprising one or more additions, deletions, substitutions of at least one amino acid will maintain their ability to be recognized by antibodies raised against non modified polypeptides.

Antibodies Raised Against the SEQ ID No 1 Sequence CGL1 Polypeptide

Fragments of the CGL1 polypeptide, in particular the polypeptides with sequences SEQ ID No 11 to SEQ ID No 13, as well as variants of the CGL1 polypeptide, more particularly polypeptides with amino acid sequences SEQ ID No 2 to SEQ ID No 10, as well as homologous peptides may be used for preparing antibodies, in particular with a view to detect the production of the normal form or on the contrary, the mutated form of the CGL1 polypeptide in a patient.

To detect the normal presence of the CGL1, the preferred antibodies according to the invention are those antibodies raised against the polypeptides with sequences SEQ ID No 11 to SEQ ID No 13.

To detect the presence of a mutated CGL1 polypeptide, the preferred antibodies are those antibodies raised against the polypeptides with sequences SEQ ID No 2 to SEQ ID No 10.

Most preferably, the antibodies for detecting the presence of a mutated CGL1 polypeptide are those antibodies raised against the mutated regions of the CGL1 polypeptide, such as previously described in detail in the disclosure, and more specifically, the C-terminal regions of the mutated CGL1 peptides which are not found in the normal CGL1 polypeptide.

According to another aspect, the antibodies according to the invention are used for:
locating in tissue and cells the CGL1 protein (comparison between cells of a control (normal protein) and those of the patients (mutated protein));
determining the physiological role of the protein, its activation and inactivation mechanisms and its natural partners.

The term “antibody” as used in the present invention more particularly refers to polyclonal or monoclonal antibodies or fragments (for example the F (ab)′₂, Fab fragments) or also to any polypeptide comprising a domain of the initial antibody recognising the target polypeptide or polypeptide fragment according to the invention.

Monoclonal antibodies can be prepared from hybridomas using the technique as described by Kohler et Milstein (1975).

The present invention also relates to antibodies raised against a polypeptide such as described hereinabove or a fragment or a variant thereof, such as produced in the trioma technique or also in the hybridoma technique such as described by Kozbor et al., (1983).

The invention also relates to Fv (ScFv) single chain antibody fragments such as described in U.S. Pat. No. 4,946,778 or also by Martineau et al. (1998).

The antibodies according to the invention also comprise antibody fragments obtained using phage banks from Ridder et al., (1995) or also humanized antibodies from Reinmann et al. (1997); Leger et al., (1997).

The antibody preparations according to the invention are useful in the immunological detection tests used for identifying the presence and/or the amount of antigens present in a sample.

An antibody according to the invention could comprise an isotopic or non isotopic detectable marker, for example a fluorescent one, or also be coupled to a molecule such as biotin, using techniques well known to the man of the art.

Another object of the invention is also to provide a method for detecting the presence of a normal or a mutated CGL1 polypeptide in a sample, said method comprising the following steps of:
a) contacting an antibody such as defined hereinabove with the sample to be tested;
b) detecting the optionally formed polypeptide-antibody complexes.

The invention is also relative to a set or a kit for detecting a normal or a mutated CGL1 polypeptide in a sample, said set or kit comprising:
a) an antibody such as defined hereinabove;
b) if need be, the reactants required for detecting the optionally formed polypeptide-antibody complexes.
   Pharmaceutical Compositions and Therapeutic Treatment Methods

Another object of the invention is also the use of a nucleic acid coding for the CGL1 polypeptide of a recombinant vector containing such a nucleic acid or of a recombinant host cell such as defined hereinabove for producing a drug for preventing or treating a pathology linked to a mutation or to a deficiency in the expression of the gene coding for the CGL1 polypeptide, preferably a pathology listed in the [symbol] SUMMARY [symbol] section of the present disclosure, including a lipodystrophia or a diabetes, as well as pharmaceutical compositions containing a nucleic acid coding for the CGL1 polypeptide, a recombinant vector containing such a nucleic acid or also a recombinant host cell such as defined hereinabove, in association with one or more physiologically compatible excipients.

The invention also relates to the use of the CGL1 polypeptide or of a fragment of the CGL1 polypeptide for producing a drug for preventing or treating a pathology linked to a mutation or a deficiency in the expression of the gene coding for the CGL1 polypeptide, preferably a pathology listed in the [symbol] SUMMARY [symbol] section of the present disclosure, including a lipodystrophia or a diabetes, as well as to pharmaceutical compositions comprising a CGL1 polypeptide or a fragment of the CGL1 polypeptide, in association with one or more physiologically compatible excipients.

The above-mentioned uses and the pharmaceutical compositions are previously described in further detail in the present specification.

According to another aspect, an object of the invention is also to provide a preventive or curative therapeutic method for treating diseases associated to lipodystrophia, preferably the congenital generalized lipodystrophia (CGL), such a method comprising a step during which a patient is administered a nucleic acid able to modify the expression of the CGL1 polypeptide in said patient, said nucleic acid being, if need be, associated with one more physiologically compatible carriers and/or excipients.

Preferably, the patient will be administered a pharmaceutical composition comprising a nucleic acid, such as defined in the present specification.

According to yet another aspect, an object of the invention is also to provide a preventive or curative therapeutic method for treating diseases associated to lipodystrophia, preferably generalized or congenital lipodystrophia (CGL), or to a diabetes, such a method comprising a step during which a patient is administered a therapeutically efficient amount of the CGL1 polypeptide in said patient, said polypeptide being, if need be, associated with one or more physiologically compatible carriers and/or excipients.

Methods for Screening Candidate Compounds of Therapeutic Interest

The demonstration according to the invention that the cgl1 gene is a CGL causal gene allows for the implementation of methods for screening candidates compounds of therapeutic interest for preventing or treating a pathology linked to a mutation or a deficiency in the expression of the gene coding for the CGL1 polypeptide, including a lipodystrophia, in particular CGL or a diabetes.

Method for Screening a Candidate Compound Interacting with the CGL1 Polypeptide

As a polypeptide involved in the development of lipodystrophia, in particular a congenital generalized lipodystrophia (CGL) or a diabetes, the CGL1 polypeptide is found to be a target polypeptide of the compounds of preventive or curative therapeutic interest of pathologies associated with CGL.

Such candidate compounds of therapeutic interest consequently behave as [symbol] ligands [symbol] of the CGL1 polypeptide. As used herein, a [symbol] ligand [symbol] means a molecule, such as a protein, a peptide, an antibody or any synthetic chemical compound able to be fixed onto the CGL1 polypeptide or one fragment thereof.

In a method for screening candidate compounds according to the invention, a biological sample or a structurally defined molecule to be tested is brought into contact with the purified CGL1 polypeptide, for example the purified recombinant CGL1 polypeptide produced by a recombinant host cell such as defined in the present disclosure, in order to form a complex between the CGL1 polypeptide or a fragment thereof and the putative ligand thereof represented by the candidate compound to be tested.

By way of illustration, to study the interaction between the CGL1 polypeptide or a fragment thereof with candidate compounds, such as molecules obtained using combining chemical methods, one will use microdialysis techniques coupled with HPLC as described by WANG et al. (1997) or the capillary electrophoresis technique as described by BUSH et al. (1997).

In other methods, candidate compounds, which may be peptides, fatty acids, lipoproteins or small molecules interacting with the CGL1 polypeptide, or a fragment of such a polypeptide, may be identified by conducting tests where the candidate compound to be tested is labeled with a detectable marker, such as a fluorescent, radioactive or enzymatic marker, then contacted with the immobilized CGL1 polypeptide, or a fragment thereof, in conditions allowing for the specific attachment of the candidate compound on the CGL1 polypeptide. After removal of the non-specifically linked molecules, the fixed molecules are detected using appropriate detection means.

The candidate compounds interacting with the CGL1 polypeptide or a fragment thereof could also be screened using techniques implementing an optical biodetector such as those described by Edwards and Leatherbarrow (1997) or also by SZABO et al. (1995).

One can also use screening systems of the double hybrid type such as those described by FIELDS & SONG (1989) or also in U.S. Pat. Nos. 5,667,973 and 5,283,173 (FIELDS et al.).

Methods for screening by double hybrid techniques can also be implemented using techniques described by HARPER et al. (1993) or also by CHO et al. (1998) or FROMONT-RACINE et al. (1997).

Another object of the invention is consequently a method for screening a candidate compound interacting with the CGL1 polypeptide or with a fragment of the CGL1 polypeptide, characterized in that it comprises the following steps of:
a) contacting the candidate compound with the CGL1 polypeptide or the fragment of the CGL1 polypeptide;
b) detecting the complexes optionally formed between the CGL1 polypeptide or the fragment of the CGL1 polypeptide, on the one hand, and the candidate compound, on the other hand.

The invention also relates to a set or a kit for screening a candidate compound interacting with the CGL1 polypeptide or with a fragment of the CGL1 polypeptide, characterized in that it comprises:
a) a CGL1 polypeptide or a fragment of the CGL1 polypeptide;
b) if need be, the reactants required for detecting the complexes optionally formed between the CGL1 polypeptide or the fragment of the CGL1 polypeptide, on the one hand, and the candidate compound, on the other hand.
   Method for Screening a Candidate Compound Modulating the Expression of the cgl1 Gene

Another object of the invention comprises a method for screening a candidate compound modulating the expression of the cgl1 gene, characterized in that it comprises the following steps of:
a) culturing a host cell expressing naturally or after a genetic recombination the cgl1 gene;
b) contacting the host cell cultured in step a) with a candidate compound to be tested;
c) determining the ability of the candidate compound to modulate the expression of the cgl1 gene by the host cell.

Preferably, step c) is a quantification step of the expression of the cgl1 gene by the host cell.

The quantification of the expression of the cgl1 gene can be indiscriminately achieved by quantification of the produced cgl1 messenger RNA or by quantification of the produced CGL1 polypeptide.

In such a case, one can use antibodies such as previously defined in the disclosure in order to quantify the CGL1 polypeptide amounts which have been produced, for example through an immunoenzymatic test or through immunoradioactivity.

In another preferred embodiment, the amount of the produced mRNA is reached through quantitative PCR amplification of the cDNA obtained after a reverse transcription of the total mRNA of the cultivated host cell, using a pair of specific primers of the cgl1 gene.

The present invention also relates to a method for screening candidate compounds able to increase or, contrarily, decrease the expression level of the cgl1 gene. Such a method makes it possible for the man of the art to select compounds exerting a regulating effect on the expression level of the cgl1 gene, a compound potentially useful as an active in pharmaceutical compositions for treating lipodystrophia predisposed or affected patients, preferably a congenital or generalized lipodystrophia (CGL) or a diabetes.

The quantitative analysis of the expression of the cgl1 gene can be conducted using matrices or [symbol] DNA chips [symbol] which could contain a plurality of nucleic acids derived from the cgl1 gene or from the corresponding cDNA.

For example, the quantitative analysis of the expression of the cgl1 gene could be conducted using a DNA chip such as described by SCHENA et al. (1995 and 1996).

Another object of the invention is also to provide a set or a kit for screening a candidate compound modulating the expression of the cgl1 gene, characterized in that it comprises:
a) a host cell expressing, naturally or after a genetic recombination, the cgl1 gene;
b) if need be, the means required for determining the ability of the candidate compound to modulate the expression of the candidate cgl1 by the host cell.

In a preferred embodiment of the above-mentioned set or kit, the latter is characterized in that the means required for determining the ability of the candidate compound to modulate the expression of the cgl1 gene by the host cell are one or more specific probes for the cgl1 gene, more specifically those probes such as previously defined in the disclosure.

Method for In vivo Screening

The invention also relates to two types of transgenic animal models, respectively, an inactivation model of the gene orthologous to cgl1 in the animal, and a model allowing for a controlled overexpression of the cgl1 gene in the animal. Both transgenic animal models are useful more particularly for screening candidate compounds of a therapeutic interest.

According to another aspect of the invention, candidate compounds modulating the expression of a nucleic acid coding for the CGL1 polypeptide according to the invention may be identified in vivo in non human transgenic animals.

Most preferably, a candidate compound of interest according to the invention comprises a compound increasing the expression level of a nucleic acid coding for the CGL1 polypeptide.

The expression “transgenic animal”, as used herein, refers to a non human animal, preferably a mammal, where one or more cells contain a heterologous nucleic acid introduced through human intervention, such as through transgenesis techniques well known to the man of the art. The heterologous nucleic acid is introduced directly or indirectly into the cell or the cell precursor, through genetic handling such as through micro-injection or infection by a recombinant virus. The heterologous nucleic acid can be integrated into the chromosome or can have the form of an extra-chromosomically replicating DNA.

According to a first aspect, a transgenic animal according to the invention comprises, in a form artificially inserted into its genome, a nucleic acid coding for a CGL1 polypeptide such as previously defined in the disclosure.

According to a second aspect, a transgenic animal according to the invention comprises, in a form artificially inserted into its genome, a nucleic acid inserted in place of the cgl1 orthologous gene and blocking the production of the corresponding polypeptide.

A. [symbol] Knock-Out> Transgenic Animal for cgl1

The invention relates to a non human transgenic animal, the somatic cells or the somatic cells and the germinal cells which have been transformed by a nucleic acid, in a targeted way, and in which genome said nucleic acid is inserted so as to inactivate the gene corresponding to cgl1, in said transgenic animal.

The gene corresponding to cgl1 is inactivated by the partial or total substitution of its sequence by said exogenous nucleic acid. In the case of a partial substitution of the sequence of the gene corresponding to cgl1, such a gene is inactivated by its sequence being interrupted by the sequence of said exogenous nucleic acid.

In other words, the inactivated gene does not express anymore, or expresses producing an inactive polypeptide.

The targeted insertion of the exogenous nucleic acid is achieved through homologous recombination, according to techniques well known to the man of the art.

Genic Inactivation of CGL1 Through Transgenesis in Mouse

In summary, a polynucleotide construct comprising the cgl1 gene modified so as to prevent the production of the CGL1 polypeptide is specifically inserted in place of the endogenous homologous gene through recombination in the 129sv mouse strain embryo cell lineage. Inserting the polynucleotide construct preferably occurs through electroporation, such as described by Thomas et al. (1987, Cell, Vol. 51:503-512).

The genomic sequence of a CGL1 including fragment has been obtained from DNA from the mice 129sv lineage, allowing to define an inactivation and screening strategy of strain embryo cells having achieved the homologous recombination. Introducing the transgene will reproduce a deletion inducing a shift in the reading framework, also described in man as causing the generalized congenital lipodystrophia syndrome.

The transfection, by the appropriate recombinant vector, of the strain embryo cells and the injection in blastocysts are achieved. The clones are then screened and the animals are genotyped.

B. [symbol] Knock-In [symbol] [symbol] Transgenic Animal for cgl1

Such a transgenic animal of the invention comprises, under a form artificially inserted into its genome, a nucleic acid coding for a CGL1 polypeptide such as previously defined in the disclosure.

Preferably, the nucleic acid coding for the CGL1 polypeptide is inserted in a targeted way in the genome of the animal, and more preferably, such a nucleic acid is inserted at the location where the gene orthologous to cgl1 is located in the genome of said animal, through homologous recombination, according to techniques well known to the man of the art.

CGL1 Controlled Overexpression in Mouse

The CGL1 controlled overexpression scheme comprises creating a transgenic mouse through targeted insertion in its genome of a copy of the CGL1 complete complementary DNA. Such a technique, called “knock-in”, also uses the homologous recombination property of the mouse 129sv lineage strain embryo cells. It comprises sub-cloning the CGL1 complete complementary DNA downstream an ubiquitous mammalian promoter or a mammalian promoter specific for a tissue, in a vector containing positive and negative selection systems, as well as the sequence of a locus allowing for an optimal expression of the target complementary DNA. The remainder of the technique is the same as the “knock-out” genetic inactivation.

Such a technique allows to control the number of copies inserted in the genome of the transgenic animal and to thereby obtain a large number of absolutely identical animals through the number of copies of the transgene. This allows for overcoming the problems encountered with the random transgene technique, wherein it is difficult to compare the results from physiological experiments because of the variable number of copies of the transgene from one animal to another.

The further advantage of such a technique is the efficiency and the control of the expression of the transgene, because it is inserted specifically in an open chromatin locus where the transcription rate is high, and it is possible to determine the location of its expression through a judicious selection of the promoter.

Another object of the invention is thus a non human transgenic animal, the somatic and/or germinal cells of which have been transformed by a nucleic acid coding for the CGL1 polypeptide.

The transcription of the cgl1 gene has been characterized by different techniques and a complete complementary DNA has been cloned in a mammalian expression vector in order to check the integrity of the polypeptide originating from such a complementary DNA. The sub-cloning of such a complementary DNA in a “knock-in” vector with an ubiquitous promoter, the transfection of strain embryo cells and the injection in a blastocyst have been conducted.

According to such a method, a non human transgenic animal, for example a mouse, is treated with a candidate molecule or substance to be tested, for example a candidate substance or molecule previously selected using an in vitro screening method such as defined hereinabove.

After a determined period, the expression level of the nucleic acid coding for the CGL1 polypeptide is determined and compared with the expression of such a nucleic acid in an identical non human transgenic animal, for example an identical transgenic mouse, which has not received the candidate molecule or substance.

The expression level of the nucleic acid coding for the CGL1 polypeptide can be determined through quantification of the corresponding messenger RNA produced by cells taken from the animal or also through detection and quantification of the CGL1 polypeptide produced in such cells, using techniques known to the man of the art or previously described in the specification.

The measurement of the levels of messenger RNA corresponds to the nucleic acid coding for the CGL1 polypeptide through hybridization of the Northern type, or also through in situ hybridization.

The measurement of the expression levels of the CGL1 polypeptide may be carried out through immunohistochemistry.

For implementing a method for in vivo screening a candidate substance or molecule modulating the expression of a nucleic acid coding for the CGL1 polypeptide, non human mammals are preferred, such as mice, rats or guinea-pigs or rabbits having their genome modified through the insertion of a polynucleotide construct comprising a nucleic acid according to the invention.

The transgenic animals according to the invention comprise the transgene, i.e. the above-mentioned polynucleotide construct, in a plurality of their somatic and/or germinal cells.

Producing transgenic animals according to the invention can be performed using conventional techniques well known to the man of the art. The man of the art could particularly refer to the production of transgenic animals, and in particular, to the production of transgenic mice, such as disclosed in U.S. Pat. No. 4,873,191 (issued on 10 Oct. 1989), U.S. Pat. No. 5,464,764 (issued on 7 Nov. 1995) and U.S. Pat. No. 5,789,215 (issued on 4 Aug. 1998), the content of such documents being incorporated into the application by reference.

Briefly, a polynucleotide construct comprising a nucleic acid coding for the CGL1 polypeptide is inserted in a type ES strain cell lineage. Inserting the polynucleotide construct occurs preferably through electroporation, such as described by Thomas et al. (1987, Cell, Vol. 51:503-512).

The cells having been subjected to the electroporation step are then screened for the presence of the polynucleotide construct (for example through selection using markers, or also through PCR or through analysis on electrophoresis gel of DNA Southern blot) so as to select positive cells having integrated the exogenous polynucleotide construct in their genome, if need be, further to a homologous recombination event. Such a technique is for example described by MANSOUR et al. (1988, Nature, Vol. 336:348-352).

Then, the positively selected cells are isolated, cloned and injected in 3,5 day mouse blastocysts, as described by BRADLEY (1987, Production and Analysis of Chimaeric Mice. In: E. J. ROBERTSON (Ed., teratocarcinomas and embryonic stem cells: A practical approach. IRL press, Oxford, page 113). Blastocysts are then introduced in a pseudo-pregnant female host animal and the embryo development is continued until full term.

According to an alternative, positively selected ES type cells are contacted with 2,5 day embryos at a 8-16 cell stage (morulae) as described by WOOD et al. (1993, Proc. Natl. Acad. Sci. USA, vol. 90: 4582-4585) or by NAGY et al. (1993, Proc. Natl. Acad. Sci. USA, vol. 90: 8424-8428), the ES cells being internalized so as to extensively colonize the blastocyst, including the cells generating the germinal lineage.

The offspring is then tested so as to determine those which have integrated the polynucleotide construct (the transgene). Through mutual crossbreeding of the transgenic animals of the first generation, homozygotic transgenic animals for transgene integration are obtained in a way known to the man of the art.

Preferably, the transgenic animals according to the invention are homozygotic for the transgene.

An object of the invention is therefore also a non human transgenic animal, the somatic and/or germinal cells of which have been transformed by a nucleic acid coding for the CGL1 polypeptide.

According to another aspect, the invention also relates to a non human transgenic animal, the somatic and/or germinal cells of which have been transformed by a nucleic acid coding for a fragment or a variant of a CGL1 polypeptide, for example one of the mutated CGL1 polypeptides CGL1 previously described herein. Such transgenic animals are useful particularly for checking the selectivity of the active candidate compounds on the non mutated CGL1 polypeptide.

According to a particular embodiment, the nucleic acid coding for the CGL1 polypeptide comprises a regulatory polynucleotide for the transcription and/or the translation under which control the coding region containing the open reading framework is located, said regulatory polynucleotide being functional in the subject transgenic animal.

According to a particular embodiment, the above-mentioned regulatory polynucleotide contains activating sequences so-called “enhancers” allowing for a high level expression of the CGL1 polypeptide in the transgenic animal's cells.

The invention also relates to recombinant host cells obtained from a transgenic animal such as described hereinabove.

Lineages of recombinant cells originating from a transgenic animal according to the invention may be cultured in the long term from any tissue of such a transgenic animal, for example through transfection of the cultures of primary cells with vectors expressing oncogens such as the SV40 big T antigen, such as described for example by CHOU (1989, Mol. Endocrinol. Vol. 3:1511-1514) and SCHAY et al. (1991, Biochem. Biophys. Acta, vol. 1072:1-7).

The invention also relates to a method for screening in vivo a candidate molecule or substance modulating the expression of a nucleic acid coding for the mutated or non mutated CGL1 polypeptide according to the invention, comprising the following steps of:
a) administering the candidate substance or molecule to a transgenic animal such as defined hereinabove;
b) detecting the expression level of the nucleic acid coding for the mutated or non mutated CGL1 polypeptide;
c) comparing the results obtained in b) with the results obtained in a transgenic animal which has not received the candidate substance or molecule.

The invention also relates to a method for in vivo screening a candidate molecule or substance modulating the expression or the activity of the CGL1 polypeptide using both animal models of the invention, comprising the following steps of:
a) administering the candidate substance or molecule to the transgenic animals corresponding to the models described hereinabove;
b) detecting the expression location and level of the CGL1 polypeptide using antibodies such as defined in the invention, characterising the clinical, biological and molecular phenotype of the model animals;
c) comparing the obtained results with those obtained in an animal which has not received the candidate substance or molecule. In particular observing a possible modification in the expression location or level of the polypeptide, or a possible reversion of the phenotype.

It also relates to a kit or set for in vivo screening a candidate molecule or substance modulating the expression or the activity of the CGL1 polypeptide, comprising:
a) a [symbol] knock-in)> or a [symbol] knock-out [symbol] transgenic animal such as previously defined;
b) if need be, the means for detecting the expression location or level of the CGL1 polypeptide.

In both cases, the animals being used for implementing the above-mentioned method are sacrificed, for example during a step d) of the method. Indeed, the above-mentioned method is a method for screening candidate compounds, with few of them being likely to exert a measurable activity in the modulation of the expression of the nucleic acid coding for the CGL1 polypeptide. Said method does not in any way aim at the therapeutic treatment of the implemented animals and should by no way be assimilated to a therapeutic or surgical treatment method applied to the human or animal body.

The invention also relates to a kit or set for in vivo screening a candidate molecule or substance modulating the expression of a nucleic acid coding for the mutated or non mutated CGL1 polypeptide, comprising:
a) a transgenic animal such as defined herein above;
b) if need be, the means for detecting the expression level of the nucleic acid coding for the mutated or non mutated CGL1 polypeptide.

The present invention will be additionally illustrated, but without any limitation, by the following examples.

### EXAMPLES

A. Materials and Methods of the Examples

A.1 Patients

31 GL affected families and 27 additional patients (isolated patients or belonging to small sized families), representing a total of 83 GL affected patients were investigated. All the patients showed a generalized lipoatrophy form and a muscle hypertrophia form. Most of them had acromegaloids, an acanthosis nigricans, a hepatomegalia, a hirsutism, an insulin-resistance with sometimes a diabetes and a hypertriglyceridemia. The biological and clinical features of some of the patients were previously documented (SEIP et al., 1996; VAN DER VORM et al. 1993; KROOK et al. K, 1994; VIGOUROUX et al., 1997; SEIP, 1959, HUSEMAN, 1978; PANZ, 1997; et VAN MALDERJEM et al., 1996). All the patients as well as their families gave their informed and enlightened assent with a view to genetic studies which were approved by an Institutional Committee.

A.2. Genotyping

The genomic DNA was obtained from white cells of peripheral blood using standard protocols (SAMBROOK et al., 1989). Genotyping markers has been performed using the LMS II-MD2 microsatellite marker panel (PERKIN Biosystems) and a DNA sequencer of the ABI 377 type provided with the GENESCAN 3.0 AND GENOTYPER software (version 2.1, commercialized by the APPLIED BISYSTEMS Corporation). For the fine mapping, new recurrent markers of the CA type were identified in the above-mentioned genomic sequences so as to be located in the studied locus and tested for their polymorphism. The possible order of such markers on the chromosome has then been determined using the irradiation hybrid panel referred to as Stanford GB3 (STEWART, 1997).

A.3 Genetic Analysis

The parametric association analysis has been conducted using algorithms such as previously described (SOBEL et al., 1996) which were implemented in the SIMWALK 2 program. The allelic frequence of the disease was established at 0.001 and a merely recessive transmission mode was assumed with a 0.99 penetrance rate for the disease genotype. The most likely haplotype configurations were also determined using the SIMWALK2 programs. Marker allele frequencies were evaluated from the data observed for families used in screening the genome. For the analysis of the additional families, the allelic frequencies of the markers were again evaluated from those family data. A new calculation of the lod score with other allelic frequency evaluations did not modify the conclusions.

A.4 Screening of Mutations

Specific primers were built for amplifying the exons and the splicing joints using the data contained in the WEB site of the MIT (http//ww-genome.wi.mit.edu/cgi-bin/primer3-ww.cgi.).

The CGL exons were screened for mutations after amplification of seven fragments (exon 1, exon 2, exon 3, exon 4, exons 5-6, exons 7-8 and exons 9-11). The sequence for those intronic primers is to be obtained on request. In patients where only very small DNA amounts were available (extracted from old hypophysial tissues and from serums), two amplifications were performed in one pass. The PCR products were purified on a SEPHADEX type column and sequenced using the [symbol] BIG DYE TERMINATOR [symbol] ABI technique.

The sequence comparisons were conducted using the PHRED PHRAP CONSED software described by EWING et al. (1998).

A.5 Analysis of mRNA Electrophoresis Gel

mRNA gels were used from various human tissues as well as the total RNA from human brains, heart and liver (CLONTECH). In addition, the total RNA was isolated from the subcutaneous and visceral abdominal adipose tissues originating from the skin and the colon after a surgery, using the QUIAGEN kit, a Neosy maxi kit.

The total RNA (20 μg) was separated on a 1% agarose gel, 2.2 M formaldehyde/MOBS and transferred on a Nylon membrane of the Hybond-N type (Amersham). The membranes were hybridized using an approximately 1700 bp probe covering exons 1 to 11 of the cgl1 gene including the starting AUG codon and the STOP codon. Such a probe was obtained through amplification of the total cDNA of brain using the advantage 2 (CLONTECH) kit and the primer couple (exon 1F: 5′-CCCC TGCAGTGGAGTCTGTA-3′ SEQ ID No 17; exon 11 R: 5′-AGTCAGGTGGGAAAGTGCTG-3′ (SEQ ID No 18).

The PCR amplification conditions were as follows: 95° C. for one minute, followed by 30 cycles of 95° C. for 30 seconds, 65° C. for 45 seconds and 72° C. for 2 minutes, and a final extension step at 72° C. for 10 minutes. The probe identity was confirmed after a new amplification with 5 internal primer pairs, followed by a partial sequencing. Quantification of the signals was performed using the Imager phosphorus device (Storm 860, Amersham Pharmacia Biotech) using the Image Quant software, Version 5.0.

A.6 Sequence Analysis

The genomic sequences included in the chromosome interval of interest were searched in the [symbol] http//www.ncbi.nlm.gov/genome/central [symbol] and [symbol] httt//hgrep.ims.u-tokyo.ac.jp/ [symbol] sites. For the homology searches, a BLAST software was used which is available on the [symbol] httt//www.ncbi.nlm.gov/cgi-bin/BLASTz,901 z,901 site. The multiple alignments were built using the CLUSTAL software available on the [symbol] http//www.infobiogen.fr/services/analyseq/cgi-bin/clustalw-out.plz,901 z,901 site. The CGL1 protein was analysed using the following computing programs: Expasy PROTEOMIC TOOLS, containing the Scan profile sofwares as well as the PROSITE software, available on the [symbol] http//www.expasy.ch [symbol] site for searching transmembrane helices with consensual pattern. The BLOCKS software was used for searching a consensus domain, available on the [symbol] http//bioinformatics.weismeinn.ac.il/blocks/ [symbol] site.

### Example 1

Identification of the CGL Causal Gene

1.1 Mapping of the Genetic Determinant for the 11q13 Chromosome Locus Disease

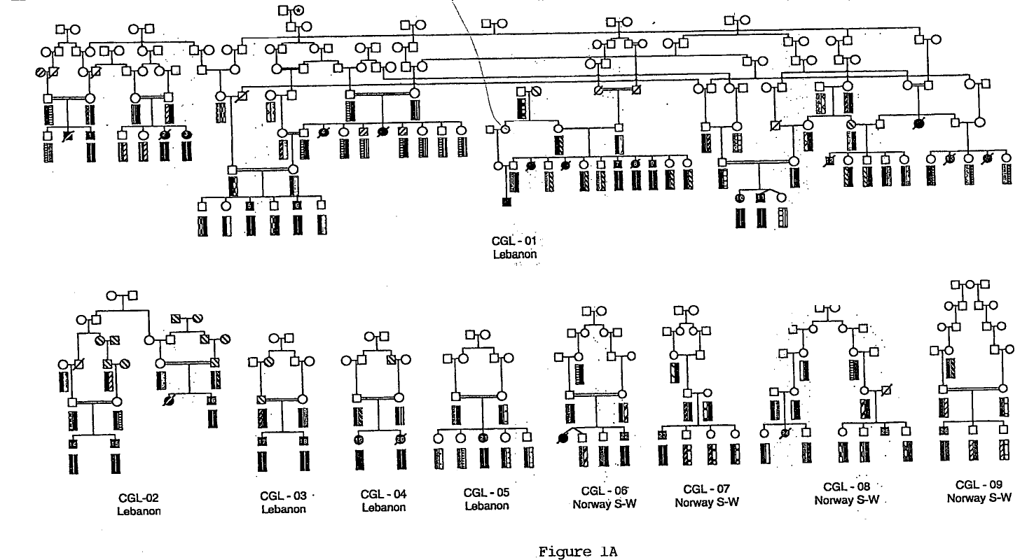

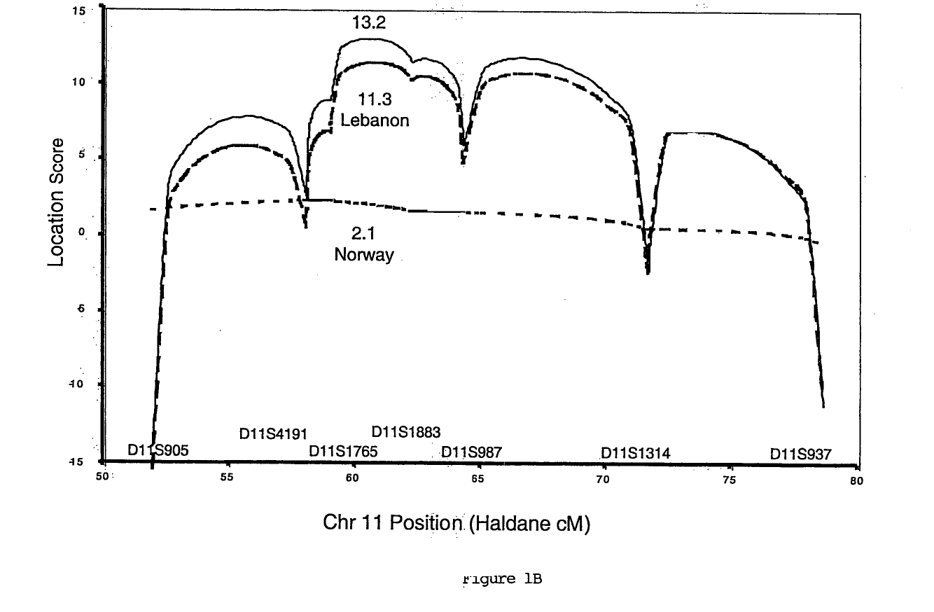

A set of Lebanese and Norwegian families was selected for the original investigations in order to locate the gene responsible for the CGL (FIG. 1A). In the five Lebanese families, the DNA was available for eighteen affected patients and 66 unaffected patients. All those families were consanguineous and three of them (CGL-01, CGL-02 et CGL-03) came from the same village, which is indicative of a geographical group. Amongst the four families from South-Western Norway (SEIP, 1996; SEIP, 1959), the DNA was available from three affected relatives and twenty-six unaffected relatives (GEDDE-DAHL et al. (1996)). Two of the families had a detected co-sanguinity and amongst two of the non consangeneous families, one of them had an affected offspring in two of its branches (GEDDE-DAHL, 1996).

A linking and homozygosity analysis covering the whole genome was initiated using the LMS 11-MD2 panel with, on average, approximately 10 cM spaced apart microsatellites. No evidence of linking has been found on the 9q34 locus (CGL1), a previously documented region (GARG et al., 1999), where the markers for the genomic screening were added (D9S164 and D9S1836) with the D9S1818 and D9S1826 markers limiting the interval. The maximal multilocus lod score in such an interval was approximately −22.5 in the Lebanese families and 0.13 in the Norwegian families.

Inspecting the genotype data for the whole genome showed two adjacent markers, markers D11S4191 and D11S987 on the 11q13 chromosome, which were homozygotic in many patients. An analysis with two additional markers, D11S1765 and D11S1883, within such an interval, confirmed those observations and showed the presence of identical alleles in all the D11S1883 marker eighteen patients from Lebanon. The evidence of a link was highly significant, with a maximum multilocus lod score of 13,2 (FIG. 1B) where most of the evidence came from the Lebanese families (lod score 11.3) while the lod score from all the Norwegian families was 2.1. No other chromosome area gave significative de lod score value. From those results, a new locus was identified, referred to as CGL2 (called CGL), within the interval of approximately 8 cM (DIB et al., 1996 limited by the D11S4191 and D11S987 markers on the 11q13 chromosome.

1.2 Fine Mapping of the Lebanese and Norwegian Families

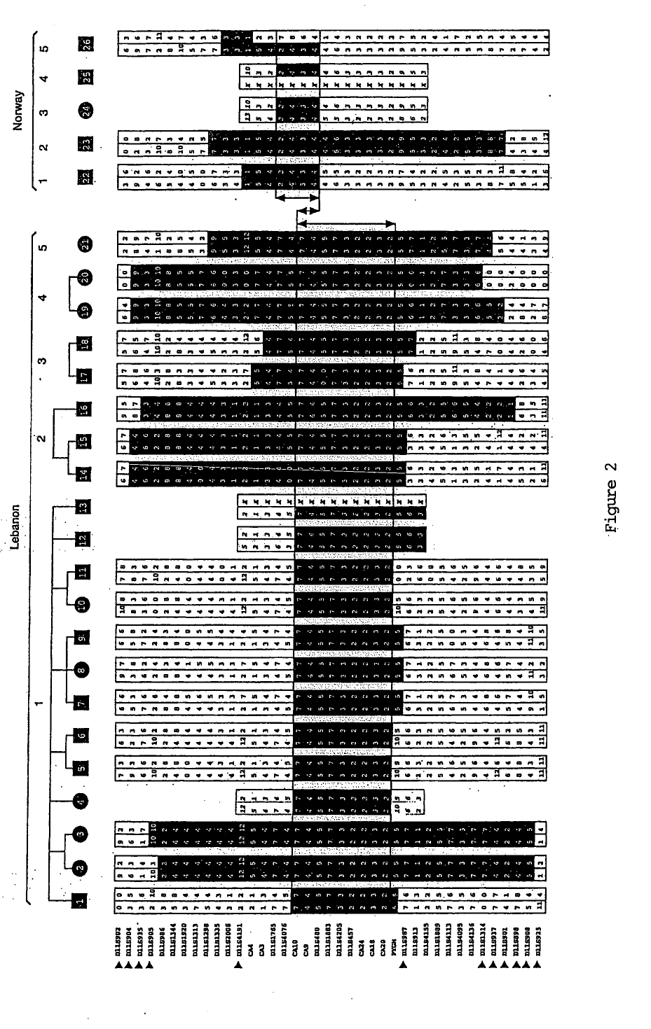

The inventors characterized twenty-five additional microsatellite markers near the CGL locus in the families, including twelve markers located in the D11S4191-D11S987 critical interval. In the Lebanese families, all the affected individuals were homozygotic for the same allele for nine contiguous markers within the D11S4076-PIGM interval, with some affected individuals being heterozygotic for the end markers as well as another marker outside the interval (FIG. 2). The presence of a common carrier allele in the five Lebanese families showed a [symbol] founding [symbol] effect in the Lebanese population, because the CGL-04 and CGL-05 families were not known for being linked to the other families. For the Norwegian population, some homozygosity was noted for the patients from the CGL-06, CGL-07 families and potentially for the CGL-08 families through deduction from relatives.

They carried the same allele for the consecutive D11S1765-D11S40766CA 110-CA9 markers, confirming the [symbol] founding [symbol] effect in those all living in the South-Western region of Norway where they could not have a common ancestor earlier than 400 years (GEDDE & DAHL et al. 1996). The affected individual from the fourth family (patient # 26, CGL-09) was heterozygotic for the markers within this interval, but with a haplotype similar to the other South-Western Norwegian families. Despite the consanguinity (consanguinity coefficient 0.00195), it is consequently possible that a second disease linked allele would have been introduced, or that recombination events leading to a small homozygosity interval would have been undetected at the resolution level of the markers.

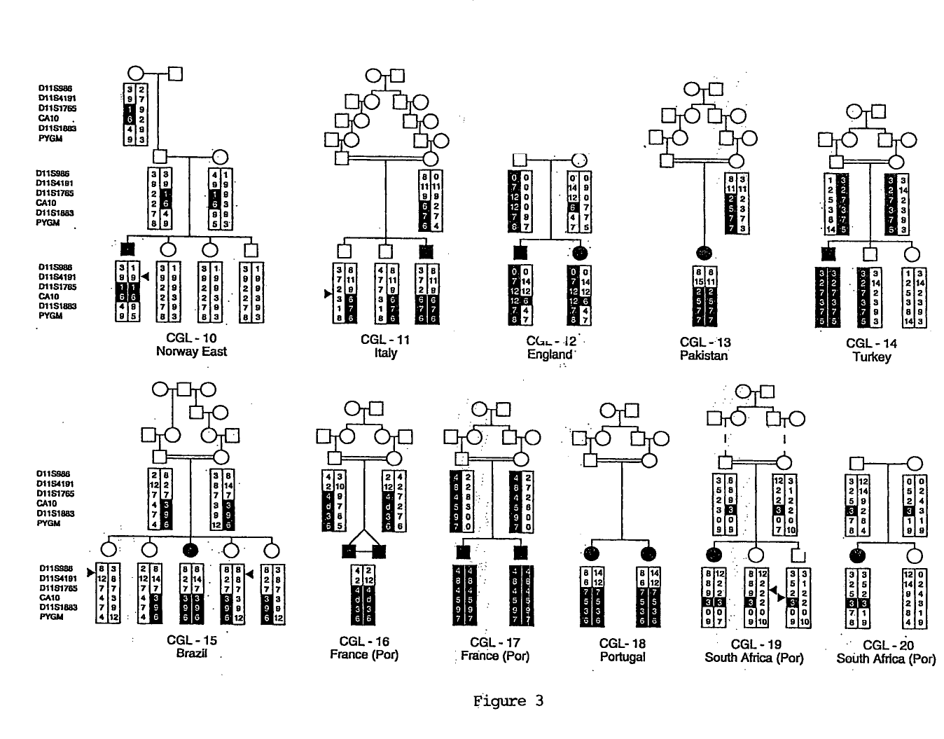

None of the unaffected children from the Lebanese and Norwegian families was homozygotic in those homozygotism intervals (FIG. 1). Since the homozygosity common region in both populations was covered at the CA10 and CA9 adjacent marker limited by the D11S4076 and D11S480 markers (FIG. 3), it has been inferred that the CGL2 locus was located in the D11S4076-D11S480 interval covering approximately 2.5 MB as determined using the physical mapping and the irradiation hybrid data. Disease linked haplotypes in the Lebanese and Norwegain families carried different alleles at both CA10 and CA9 microsatellite markers, suggesting that different mutations could be present in both geographical groups.

1.3 Families From Various Ethnic Origins Linked to the 11q13 Locus

Patients and relatives in twenty-two additional families from various ethnic origins were tested with six markers covering an approximately 10 cM interval (DIB et al. 1996; STEWART et al., 1997) at the 11q13 locus (D11S986, D11S4191, D11S1765, CA10, D11S1883, PYGM). The homozygosity and the haplotype share in the affected and the unaffected offspring suggested a co-segregation with the disease in eleven families where six of them were from Portuguese origin (FIG. 3). All the patients from those families were diagnosed as being CGL affected. In the eleven remaining families, the disease did not segregate with the 11q13 locus, for one or more of the following reasons:
1. the consanguineous relatives did not share any common alleles for the markers in the target region;
2. the patients born from consanguineous relatives were found heterozygotic;
3. an affected child had the same haplotype as the normal offspring; or
4. the affected offspring within the same family inherited different haplotypes.
   1.4. Identification of a Deletion in the Human Gene Homologous to the Gng31g Murine Gene

By screening the data bases in the D11S4076-D11S480 interval, twenty-six potential genes were identified, from gDNA, cDNA or EST sequences. The genomic structure in those 26 genes was determined through search for similarity with the human genomic sequences and specific primers were designed in order to analyze the coding region and the flanking intronic links. The sequence analysis for those twenty-six genes did not show any significant molecular alteration in a set of seven individuals including four patients from the 11q13 linked families. During those studies, patients (monozygotic twins) from one of the families where the disease was potentially linked to the 11q13 (CGL-16) locus were found to be homozygotic for one nul allele at the CA10 microsatellite marker (FIG. 3). Other genes located in the vicinity and other markers, including the CA9 microsatellite marker lying at 100 kb of the CA10 marker, were correctly amplified from those patients' DNA. Consequently, it was inferred that the patients were likely to carry a deletion in a DNA segment containing the CA 10 marker. The analysis of data bases of public sequences allows for the identification of an unknown mRNA (AF052149) corresponding to a new human gene, the cgl1 gene, which is contained in the AP001458 BAC sequence from which the CA10 marker was originally isolated. cgl1 is homologous to the Gng31g murine gene (for “Gng3-linked gene”, described by DOWNES et al., 1998), lying in an region maintained between the 19 murine chromosome and the 11 q human chromosome.

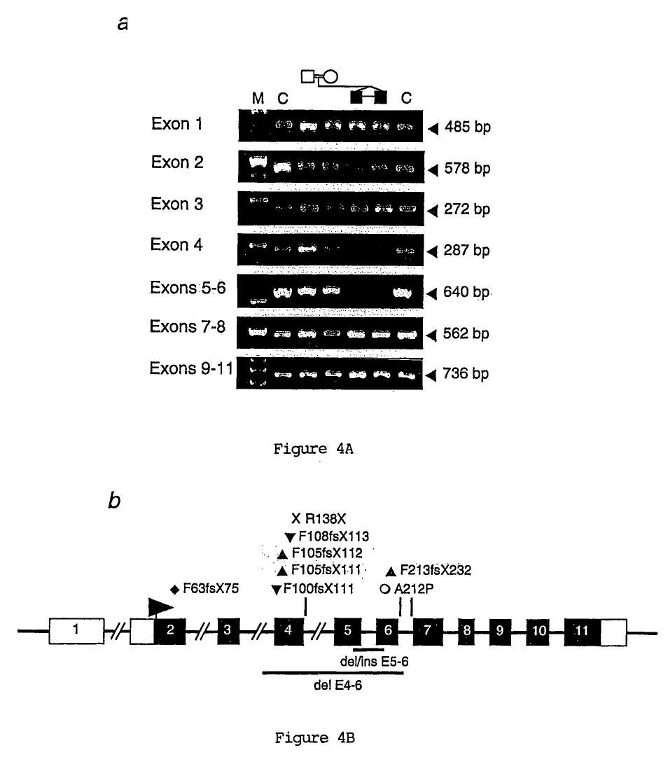

The inventors determined that the human gene, covering at least 14 kb of genomic sequences, contains 11 exons. The open reading framework (ORF) starts in exon 2 and the intron-exon links show a high similarity with the consensus sequences in splicing sites 5′ and 3′. The amplification of those exons in patients from the CGL-16 family showed the presence of a deletion including exons 4, 5 and 6 of the gene (del Ex4-6) (FIG. 4A). Based on the extent of the deletion and the AP001458 available sequence, the CA10 marker could be located either in intron 4 or in a small region of intron 3.

### Example 2

Mutation Screening in the cgl1 Gene

The cgl1 gene has been sequenced in patients from all the previously studied families. Additionally, 27 patients were analysed including 16 isolated patients and 11 patients coming from small families which were not previously studied. Twelve different molecular alterations were found amongst 41 patients coming from 22 families. Those molecular alterations include a 258 bp deletion (del E5-6), six small insertions and/or deletions lower than 10 bp, and five substitutions of one single nucleotide (FIG. 4b and table 2). Eleven of those mutations result in a change in the reading framework, the introduction of an early STOP codon or could affect a sequence in a splicing site, all the mutations being potentially null mutations. The patients, who showed an early lipoatrophy form, were found to be homozygotic for a specific mutation, or composite heterozygotic for distinct mutations. Those variants were found at the heterozygotic state in the relatives and some unaffected offspring, but were absent from the 96 controls of the CEPH families, indicating that those mutations were likely to represent disease associated mutations according to a recessive transmission mode.

Five mutations were found in more than one pedigree. As this was expected as a result of the haplotype data, the nineteen patients (a newly-born patient being added for mutation analysis) of the Lebanese families in FIG. 1 were found to be homozygotic for the same mutation, a 5 bp deletion in exon 4, which induces a change in the open reading framework after the 105 amino acid and introduces an early STOP codon at position 111 (F105 fsXW111). Similarly, the five patients (three patients added for the mutation analysis) from the three South-West Norway families (CGL-06, CGL-07, CGL-08) were homozygotic for one false sense mutation in exon 6 leading to a substitution of the alanine residue in position 212 with a proline residue (A212P). Again, as expected in the haplotype analysis, the patient originating from the fourth Norwegian family (patient # 26, CGL-09) was a heterozygotic composite with the A212P mutation inherited from his father and a different mutation inducing a change in the reading framework (F63fsX75) coming from his mother. It is interesting to note that the last F63fsX75 mutation was also found in the CGL-10 families from Easter Norway, the CGL-11 families from Italy and the CGL-12 families from the United-Kingdom. Two consanguineous families from Portuguese origin (CGL-17 and CGL-18) carried the same R138X mutation. Also, two families from Portuguese origin living in South Africa (CGL-19 and CGL-20) had a common mutation (F108fsX113). When the same mutation was present in the apparently non related families, the disease associated haplotypes carried the same allele at the CA10 marker, suggesting the occurrence of the same mutational events (FIG. 3).

In three cases, the disease associated chromosomes carried single variants at the level of or in the vicinity of the splicing site. The variant present in the CGL-14 Turkish family comprises a transition at the first base of intron 4 belonging to a nucleotide consensus sequence strictly maintained for the RNA (splicing donor site, HODGES et al., 1994). The other variants affect a located nucleotide after the consensus sequence of each splicing accepting site (intron 4: third base before exon 5; CGL-40) or the splicing donor site (intron 6: 5^{th} base; CGL-12 father's allele). Both those nucleotides are generally maintained (about 80%) at the link of the splicing sites, and mutations in those nucleotides in other genes were identified as inducing the excision of an exon or the activation of splicing cryptic sites (HODGES et al., 1994; BIENVENU et al., 1994). No other variant was detected in the exons or in the flanking intronic regions in those individuals. Thus, the alterations were very probably splicing mutations leading to a dismantling of the protein.

The systematic sequencing of the eleven exons in the cgl1 gene did not show any mutation in the patients originating from the eleven families which were not linked to the 11q13 chromosome locus, with the exception of the second affected individual from the CGL-28 family. This patient shows a false sense heterozygotic mutation, which is absent in his affected brother and sister and which, consequently, does not segregate with the disease. Also, no mutation was found within the remaining twenty-five patients, whether isolated or originating from small families, being affected either by the congenital form (14 patients) or by the delayed form (11 patients) of the disease.

These results indicate that a CGL1 distinct gene or other non genetic factors are responsible for the disease in these patients.

### Example 3

Expression Profile of cgl1

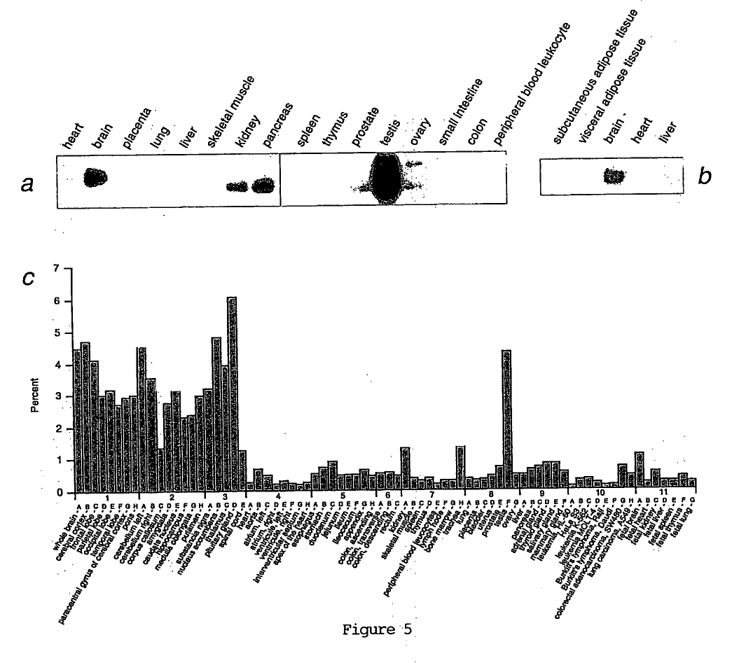

The specific expression of CGL1 tissue has been searched through mRNA hybridization (Northern Blots and Dot Blot) using a probe covering exons 1 to 11. The [symbol] Northern Blot [symbol] [symbol] analysis showed two RNA species at about 2,4 kb and about 1,8 kb expressed in a variable way in all the tissues, as well as an intermediate additional transcript of about 2 kb found as single transcript in the brain and in association with the other transcripts in the testicles (FIG. 5). The expression levels vary depending on the tissues, these levels being high in the brain and the testicles, intermediate in the (visceral and subcutaneous) adipose tissues, the pancreas, the kidney, the skeleton muscle, the liver, the ovary, the prostate and the heart and low in the remainder tissues. The hybridization analysis on [symbol] Dot Blot [symbol] gels conducted with numerous human tissues, the amounts having been adjusted to the expression level of eight ubiquitous genes [symbol] house keeping genes [symbol] , showed that the CGL1 expression in the foetal tissues and the high expression levels in most of the regions in the CNS appeared in particular in the hypophysis.

### Example 4

CGL1 Polypeptide

The cgl1 gene codes for a protein with 398 amino acids having a 43.8 kD calculated mass also referred to as [symbol] SEIPIN [symbol] .

The amino acid sequence shows a high identity with the protein coded by mouse Gng31g (87% identity) and a partial homology with a drosophila melanogaster protein (40% identity between residues 22 and 257).

The analysis did not show any similarity with other known proteins.

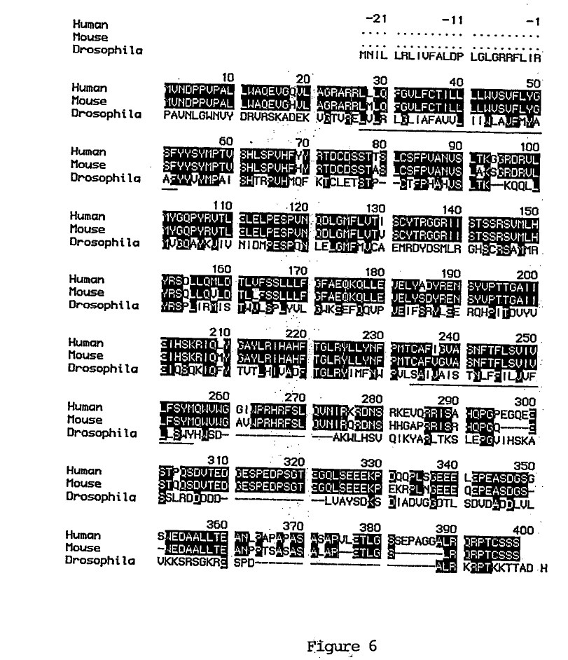

However, the results suggest that the CGL1 protein is a membrane protein having at least two transmembrane domains (FIG. 6).

TABLE 2
Small mutations found in the cgl1 gene

| | | | | | | Patients | |
|---|---|---|---|---|---|---|---|
| Gene | Nucleotide | Change in | Families | | | | Age of |
| location | alteration | amino acid | # | Origin | Consanguinity | State | occurrence |
| Exon 2 | 536delCCinsGGA | F63fsX75 | 09 | Norway (SW) | + | het | Birth |
| | ″ | ″ | 10 | Norway (East) | − | hom | Birth |
| | ″ | ″ | 11 | Italy | + | hom | <9 months |
| | ″ | ″ | 12 | United Kingdom | − | het | Birth |
| Exon 4 | 646InsAA | F10fsX111 | 15 | Brazil | + | hom | 0.5-1 month |
| | 659delGT | F10fsX112 | 36 | France | − | het^{b} | 3-6 months |
| | 859delGTATC | F10? X111 | 01 | Lebanon | + | hom | 0-1 month |
| | ″ | ″ | 02 | Lebanon | + | hom | Birth |
| | ″ | ″ | 03 | Lebanon | + | hom | Birth |
| | ″ | ″ | 04 | Lebanon | + | hom | Birth |
| | ″ | ″ | 05 | Lebanon | + | hom | Birth |
| | 669InsA | F108fsX113 | 19 | South Africa (Por) | + | hom | Birth |
| | ″ | ″ | 20 | South Africa (Por) | − | hom | Birth |
| | 758C→T | R138X | 16 | France (Por) | + | hom | Birth |
| | | | 17 | Portugal | + | hom | ˜6 months |
| Intron 4 | 638/+1G→A | ΔV98-S146 | 14 | Turkey | + | hom | Birth |
| | (excision Exon 4) | (deletion 48 aa) | | | | | |
| Exon 6 | 978G→C | A212P | 06 | Norway (W.S.) | + | hom | Birth |
| | ″ | ″ | 07 | Norway (W.S.) | − | hom | Birth |
| | ″ | ″ | 08 | Norway (W.S.) | − | hom | Birth |
| | ″ | ″ | 09 | Norway (W.S.) | + | het | Birth |
| | 980delC | P213fsX232 | 13 | Pakistan | + | hom | Birth |
| Intron 6 | 1015/+5G→A | Splicing | 12 | United Kingdom | − | het | Birth |
| | | mutation? | | | | | |
| | 1016/−3C→A | F224 to Y225- | 40 | France (Tur) | + | hom | Birth |
| | (excision Exon 7) | Q271 fsX288 | | | | | |

a: the nucleotide numbering starts at nucleotide in position 346 of the SEQ ID N0 4 sequence, such nucleotide being numbered 1 in the table
^{b}the patient from the CGL35 family is a composite heterozygote showing a deletion (258 bp), an insertion (12 bp) in exons 5-8
Het: composite heterozygotic;
hom: homozygotic for the mutation,
Por, Portuguese,
Tur: from Turkish origin

TABLE 3
Nucleic acid and amino acid sequences

| SEQ ID N^{o} | Meaning |
|---|---|
| | Polypeptides |
| 1 | CGL1 polypeptide |
| 2 | F63fsX75 mutated polypeptide |
| 3 | F100fsX111 mutated polypeptide |
| 4 | F105fsX112 mutated polypeptide |
| 5 | F105fsX111 mutated polypeptide |
| 6 | F108fsX113 mutated polypeptide |
| 7 | R138X mutated polypeptide |
| 8 | Mutated polypeptide (exon 4 deletion) Δ V99-S146 |
| 9 | A212P mutated polypeptide |
| 10 | F213fsX232 mutated polypeptide |
| 11 | CGL1 peptidic fragment |
| 12 | CGL1 peptidic fragment |
| 13 | CGL1 peptidic fragment |
| | Nucleic acids |
| 14 | DNAc coding for the CGL1 polypeptide |
| 15 | CA10/F primer |
| 16 | CA10/R primer |
| 17 | Exon 1 primer |
| 18 | R-1587 primer |

### REFERENCES

Altschul S. F. et al, J. Mol. Biol. 1990 215: 403-410
Altschul S. F. et al, Nucleic Acids Res. 1997 25: 3389-3402 Applebaum-Boden, JCI 97, 1996
Ausubel et al., 1989, Current Protocols in Molecular Biology, Green Publishing Associates and Wiley Interscience, N.Y.
Beard et al., Virology 75 (1990) 81
Beaucage et al., Tetrahedron Lett 1981, 22: 1859-1862
Becq et al. Daynal of Biological Chemistry vol 272, no 5 pages 2695-2699, 1997
Beg et al PNAS 87 p3473 1990
Bender et al., J. Virol. 61 (1987) 1639
Bernstein et al. Genet. Eng. 7 (1985) 235
Boulianne et al; Nature 312:643 (1984)
Berardinelli, W. An undiagnosed endocrinometabolic syndrome: report of two cases. J Clin Endocrinol Metab 14, 193-204 (1954).
Breakfield et al., New Biologist 3 (1991) 203
Brown E L, Belagaje R, Ryan M J, Khorana H G, Methods Enzymol 1979; 68:109-151.
BUSH et al., 1997; J. Chromatogr., vol. 777: 311-328.
Cabilly et al., U.S. Pat. No. 4,816,567
Chen and Kwok Nucleic Acids Research 25:347-353 1997
Chen et al. Proc. Natl. Acad. Sci. USA 94/20 10756-10761,1997
Chen et al., 1987, Mol. Cell. Biol., 7: 2745-2752.
CHO R J et al., 1998, Proc. Natl. Acad. Sci. USA, vol. 95 (7): 3752-3757
Chomczynski, P. and Sacchi, 1987, Anal. Biochem., 162, 156-159.
Desbois-Mouthon, C. et al. Lipoatrophic diabetes: genetic exclusion of the insulin receptor gene. J Clin Endocrinol Metab 80, 314-319 (1995).
DeRisi J. et al., 1997, Science, 278, 680-686.
Dib C. et al., 1996, Nature, 380: 152-154.
Dib, C. et al. A comprehensive genetic map of the human genome based on 5,264 microsatellites. Nature 380, 152-154 (1996).
Downes, G. B., Copeland, N. G., Jenkins, N. A. & Gautam, N. Structure and mapping of the G protein gamma3 subunit gene and a divergently transcribed novel gene, gng31g. Genomics 53, 220-230 (1998).
Eckenrode, S. et al. Fine-mapping of the type 1 diabetes locus (IDDM4) on chromosome 11q and evaluation of two candidate genes (FADD and GALN) by affected sibpair and linkage-disequilibrium analyses. Hum Genet 106, 14-18 (2000).
EDWARDS & LEATHERBARROW, 1997, Analytical Biochemistry, vol. 246:1-6
Felgner et al., PNAS 84 (1987) 7413
Flotte et al., 1992, Am. J. Respir. Cell Mol. Biol., 7: 349-356.
Forsell Y. et al., 1997, Biol. Psychiatry, 42: 898-903.
Fraley et al., 1979, Proc. Natl. Acad. Sci. USA, 76: 3348-3352.
Fraley et al., J. Biol. Chem. 255 (1980) 10431
FROMONT-RACINE M et al., 1997, Nature Genetics, vol. 16(3): 277-282.
Francis et al., 1995.
Fromont-Racine et al, 1993. Nucleic Acid Res. 21(7):1683-1684
FIELDS & SONG, 1989, Nature, Vol. 340: 245-246.
Fuller S. A. et al., 1996, Immunology in Current Protocols in Molecular Biology, Ausubel et al.
Gopal, 1985, Mol. Cell. Biol., 5:1188-1190.
Graham et al., 1973, Virology, 52: 456-457.
Graham et al., J. Gen. Virol. 36 (1977) 59
Garg, A. et al. A gene for congenital generalized lipodystrophy maps to human chromosome 9q34. J Clin Endocrinol Metab 84, 3390-3394 (1999).
Galli, J. et al. Genetic analysis of non-insulin dependent diabetes mellitus in the GK rat. Nat Genet 12, 31-37 (1996).

Gauguier, D. et al. Chromosomal mapping of genetic loci associated with non-insulin dependent diabetes in the GK rat. Nat Genet 12, 38-43 (1996).

Gedde-Dahl, T. Jr. et al. Genetics of the Berardinelli-Seip syndrome (congenital generalized lipodystrophy) in Norway: epidemiology and gene mapping. Berardinelli-Seip Study Group. Acta Paediatr Suppl 413, 52-58 (1996).

Haff L. A. and Smirnov I. P., Genome Research, 7:378-388, 1997
Ham, Methods Cell. Biol. 21a (1980) 255
Hames B D and Higgins S J, 1985, “Nucleic acid hybridization: a practical approach”, Hames and Higgins Ed., IRL Press, Oxford.
Hammeling et al., in: Monoclonal Antibodies and T-Cell Hybridomas, Elsevier, N.Y., pp. 563-681 51981
Harland et al., 1985, J. Cell. Biol., 101: 1094-1095.
HARPER J W et al., 1993, Cell, vol. 75: 805-816.
Hoeg PNAS 93p 11448, 1996
Horvath S. et al., 1998, Am. J. Hum. Genet., 1998, 63: 1886-1897.
Houben Weyl, 1974, in Meuthode der Organischen Chemie, E. Wunsch Ed., Volume 15-I et 15-II,
Hegele, R. A. Familial partial lipodystrophy: a monogenic form of the insulin resistance syndrome. Mol Genet Metab 71, 539-544 (2000).
Huygen et al., 1996, Nature Medicine, 2(8):893-898
Kaneda et al., Science 243 (1989) 375
Kim et al. Genomics (1996), 34:213-218)
Koch Y., 1977, Biochem. Biophys. Res. Commun., 74:488-491
Köhler et al, Eur. J. Immunol. 6:511 (1976)
Köhler et al, Eur. J. Immunol. 6:292 (1976)
Köhler et al, Nature 256:495 (1975)
Kohler G. and Milstein C., 1975, Nature, 256: 495.
Kozbor et al., 1983, Hybridoma, 2(1):7-16.
Krook, A. et al. Molecular scanning of the insulin receptor gene in syndromes of insulin resistance. Diabetes 43, 357-368 (1994).
Lawrence, R. D. Lipodystrophy and hepatomegaly with diabetes, lipemia and other metabolic disturbances. Lancet 1, 724-741 (1946).
Lander and Schork, Science, 265, 2037-2048, 1994
Lander, E. S. & Botstein, D. Homozygosity mapping: a way to map human recessive traits with the DNA of inbred children. Science 236, 1567-1570 (1987).
Langmann T. et al., 1999, Biochem. Biophys. Res. Comm., 257: 29-33.
Leger O J, et al., 1997, Hum Antibodies, 8(1): 3-16
Levrero et al., Gene 101 (1991
Luciani M. F. et al., 1994, Genomics, 21:150-159.
Lyer V. et al., 1999, Science, 283: 83-87.
MacKinnon et al Gene 19 p33 1982
Maniatis et al. Molecular cloning: A Laboratory Manual, Cold Spring Harbor Press, Cold Spring, N.Y. 1982
Marcil M. et al., 1999, Artreriosclerosis Throbosis and Vascular Biology, 17: 1813-1821.
Martineau P, Jones P, Winter G, 1998, J Mol Biol, 280(1):117-127
McCormick, BioTechnology 3 (1985) 689
McGrory et al (Virology, 163, 614, 1988
McLaughlin B A et al., 1996, Am. J. Hum. Genet., 59: 561-569.
Merrifield R B, 1965a, Nature, 207(996): 522-523.
Merrifield R B., 1965b, Science, 150(693): 178-185.
Morrison et al., EP 173494
Morrison, Science 229:1202 (1985)
Moller, D. E. & O'Rahilly, S. Syndromes of severe insulin resistance: clinical and pathophysiological features. in Insulin resistance (ed. Moller, D. E.) 49-81 (Wiley and Sons, New York, 1993).
Narang S A, Hsiung H M, Brousseau R, Methods Enzymol 1979; 68:90-98
Neuberger et al., Nature 314: 268 (1985)
Neuberger et al., WO 8601533
Nickerson D. A. et al., Genomics, 1992, 12: 377-387.
Nicolau C. et al., 1987, Methods Enzymol., 149:157-76.
Oi et al., Biotechnique 4:214 (1986)
Okayama (Mol Cell. Biol. 7: 2745, 1987
Okazawa, H. et al. No coding mutations are detected in the peroxisome proliferator-activated receptor-gamma gene in Japanese patients with lipoatrophic diabetes. Diabetes 46, 1904-1906 (1997).
Pagano et al., J. Virol. 1 (1967) 891
Potter et al., 1984, Proc Natl Acad Sci USA.;81(22):7161-5
Reimann K A, et al., 1997, AIDS Res Hum Retroviruses. 13(11): 933-943
Remaley et al. ATVB 17,1813,1997 Chen et al Mol Cell Biol 7 p2745 1987
Ridder R, Schmitz R, Legay F, Gram H, 1995, Biotechnology (N Y), 13(3):255-260
Rigaud et al. Biochimica et Biophysica Acta 1231 (1995) 223-246
Robinson et al., WO 8702671
Rust S. et al., Nature Genetics, vol. 20, Septembre 1998, pages 96-98
Sambrook, J. Fritsch, E. F., and T. Maniatis. 1989. Molecular cloning: a laboratory manual. 2ed. Cold Spring Harbor Laboratory, Cold spring Harbor, N.Y.
Samulski et al., 1989, J. Virol., 63: 3822-3828.
Sanchez-Pescador R., 1988, J. Clin. Microbiol., 26(10):1934-1938
Sham P. C. et al., Ann. Hum. Genet., 1995, 59: 323-336.
Seip, M. & Trygstad, O. Generalized lipodystrophy, congenital and acquired (lipoatrophy). Acta Paediatr Suppl 413, 2-28 (1996).
SCHENA et al., Science, vol. 270: 467-470, 1995.
SCHENA et al., 1996, Proc. Natl. Acad. Sci. USA, vol. 93 (20): 10614-10619.
Sternberg N. L., 1992, Trends Genet., 8: 1-16.
Sternberg N. L., 1994, Mamm. Genome, 5: 397-404.
Strautnieks S. S. et al., 1998, Nature Genetics, 20: 233-235.
Silver, K. et al. Molecular scanning of beta-3-adrenergic receptor gene in total congenital lipoatrophic diabetes mellitus. J Clin Endocrinol Metab 82, 3395-3398 (1997).

Tacson et al., 1996, Nature Medicine, 2(8):888-892.
Taniguchi et al., EP 171496
Tur-Kaspa et al, 1986, Mol. Cell. Biol., 6: 716-718.
Urdea M. S., 1988, Nucleic Acids Research, 11: 4937-4957
Urdea M S et al., 1991, Nucleic Acids Symp Ser., 24: 197-200.
Vaisman J B C 270 p12269, 1995
Van der Vorm, E. R. et al. Patients with lipodystrophic diabetes mellitus of the Seip-Berardinelli type, express normal insulin receptors. Diabetologia 36, 172-4. (1993).
Seip, M. Lipoatrophy and gigantism with associated endocrine manifestations: a new diencephalic syndrome? Acta Paediatr Scand 48, 555-574 (1959).
Stewart, E. A. et al. An STS-based radiation hybrid map of the human genome. Genome Res 7, 422-433 (1997).
Vigouroux, C. et al. Genetic exclusion of 14 candidate genes in lipoatropic diabetes using linkage analysis in 10 consanguineous families. J Clin Endocrinol Metab 82, 3438-3444 (1997).
Vigouroux, C. et al. Human peroxisome proliferator-activated receptor-gamma2: genetic mapping, identification of a variant in the coding sequence, and exclusion as the gene responsible for lipoatrophic diabetes. Diabetes 47, 490-492 (1998).
Vigouroux, C. et al. Lamin A/C gene: sex-determined expression of mutations in Dunnigan-type familial partial lipodystrophy and absence of coding mutations in congenital and acquired generalized lipoatrophy. Diabetes 49, 1958-1962 (2000).
Wands et al., Gastroenterology 80:225-232 (1981)
WANG et al., 1997, Chromatographia, vol. 44: 205-208.
Xiong M. et al., 1999, Am. J. Hum. Genet., 64: 629-640.
Yamon et al. Blood vol 90, no 8 pages 2911-2915, 1997
SZABO et al., 1995, Curr. Opin. Struct. Biol., vol. 5 (5): 699-705.

1. A nucleic acid encoding for a CGL1 polypeptide or for a fragment or a variant of the CGL1 polypeptide with a SEQ ID No 1 amino acid sequence.

2. A nucleic acid according to claim 1, characterized in that the variant of the CGL1 polypeptide is selected amongst polypeptides with amino acid sequences from SEQ ID No 2 to SEQ ID No 10.

3. A nucleic acid according to claim 1, characterized in that the fragment of the CGL1 polypeptide is selected amongst polypeptides with amino acid sequences from SEQ ID No 11 to SEQ ID No 13.

4. A nucleic acid according to claim 1, characterized in that it comprises the polynucleotide ranging from the nucleotide in position 345 up to the nucleotide in position 1541 of the SEQ ID No 14 nucleotide sequence.

5. A nucleic acid according to claim 1, characterized in that it comprises the cgl1 gene contained in the BAC vector referred to as RP 11-831H9 with its nucleotide sequences being referenced in the GenBank data base under the access number No AP001458, or No AC 090306.

6. A probe or a nucleotide primer that hybridises specifically with a nucleic acid according to any one of claims 1 and 3.

7. (canceled)

8. A probe or a nucleotide primer hybridising specifically with the mutated cgl1 gene.

9. A probe or a nucleotide primer hybridizing specifically with the mutated cgl1 gene, which further hybridizes specifically with a nucleic acid according to claim 2.

10. A probe or nucleotide primer according to claim 8, characterized in that it hybridizes specifically with the cgl1 gene carrying a mutation selected amongst the F63fsX75, F100fsX111, F105fsX112, F105fsX111, F108fsX113, R138X, A212P, F213fsX232, del/ins E5-6 et del E4-6 mutations.

11. A nucleotide primer couple for detecting a mutation within the cgl1 gene, characterized in that it is selected amongst the nucleotide sequences SEQ ID No 15 and SEQ ID NO 16, and SEQ ID No 17 and SEQ ID No 18.

12. A method for detecting a mutation within a nucleic acid coding for the CGL1 polypeptide, characterized in that it comprises the following steps of:
a) contacting one or more nucleotide probes according to any one of claims 8 to 10 with the sample to be tested;
b) detecting the complex optionally formed between the probe(s) and the nucleic acid present in the sample.

13. A detecting method according to claim 12, characterized in that the probe(s) is/are immobilized on a substrate.

14. A set or a kit for detecting a mutation in the cgl1 gene, characterized in that it comprises:
a) one or more nucleotide probes according to any one of claims 8 to 10;
b) if need be, the reactants required for the hybridization reaction.

15. A method for detecting a mutation in a nucleic acid coding for the CGL1 polypeptide, characterized in that it comprises the following steps of:
a) contacting the sample to be tested with one or more primers according to any one of claims 8 to 11;
b) detecting the amplified nucleic acids.

16. A set or a kit for detecting a mutation in a nucleic acid coding for the CGL1 polypeptide, characterized in that it comprises:
a) one or more nucleotide primers according to any one of claims 8 to 11;
b) if need be, the reactants agents required for the amplification reaction.

17. A recombinant vector comprising a nucleic acid according to any one of claims 1 to 5.

18. A recombinant host cell which is transfected or transformed by a nucleic acid according to any one of claims 1 to 5 or by a recombinant vector comprising a nucleic acid according to any one of claims 1 to 5.

19. A preventive or curative therapeutic method which comprises administering to a subject prone to or afflicted with a lipodystrophia, a Lawrence syndrome or obesity an effective amount of a nucleic acid according to any one of claims 1 and 3 to 5, a recombinant vector comprising a nucleic acid according to any one of claims 1 and 3 to 5, or a recombinant host cell which is transfected or transformed by a) a nucleic acid according to any one of claims 1 and 3 to 5 or by b) a recombinant vector comprising a nucleic acid according to any one of claims 1 and 3 to 5.

20. A preventive or curative therapeutic method which comprises administering to a subject prone to or afflicted with diabetes or an insulin-resistant syndrome an effective amount of a nucleic acid according to any one of claims 1 and 3 to 5, a recombinant vector comprising a nucleic acid according to any one claims 1 and 3 to 5, or a recombinant host cell which is transfected or transformed by a) a nucleic according to any one of claims 1 and 3 to 5 or by b) a recombinant vector comprising a nucleic acid according to any one of claims 1 and 3 to 5.

21. A pharmaceutical composition for preventing or treating a lipodystrophia, a Lawrence syndrome or obesity comprising a nucleic acid, a recombinant vector comprising a nucleic acid, or a recombinant host cell which is transfected or transformed by a nucleic acid or by a recombinant vector comprising a nucleic acid, in association with one or more physiologically compatible excipients, and wherein the nucleic acid is a nucleic acid according to any one of claims 1 and 3 to 5.

22. A pharmaceutical composition for preventing or treating diabetes or an insulin-resistant syndrome, comprising a nucleic acid, a recombinant vector comprising a nucleic acid, or a recombinant host cell which is transfected or transformed by a nucleic acid or by a recombinant vector comprising a nucleic acid, in association with one or more physiologically compatible excipients, and wherein the nucleic acid is a nucleic acid according to any one of claims 1 and 3 to 5.

23. A polypeptide characterized in that it is the CGL1 polypeptide with a SEQ ID No 1 sequence or a fragment or a variant of the CGL1 polypeptide.

24. A polypeptide according to claim 23, characterized in that the variant of the CGL1 polypeptide is selected amongst the polypeptides with amino acid sequences from SEQ ID No 2 to SEQ ID No 10.

25. A polypeptide according to claim 23, characterized in that the fragment of the CGL1 polypeptide is selected amongst the polypeptides with amino acid sequences from SEQ ID No 11 to SEQ ID No 13.

26. A preventive or curative therapeutic method which comprises administering to a subject prone to or afflicted with a lipodystrophia, a Lawrence syndrome or obesity an effective amount of a drug comprising CGL1 polypeptide with the SEQ ID No. 1 sequence.

27. A preventive or curative therapeutic method which comprises administering to a subject prone to or afflicted with diabetes or an insulin-resistant syndrome an effective amount of a drug comprising a CGL1 polypeptide with the SEQ ID No. 1 sequence.

28. A pharmaceutical composition for preventing or treating a lipodystrophia, a Lawrence syndrome or obesity, comprising a CGL1 polypeptide with a SEQ ID No 1 sequence, in association with one or more physiologically compatible excipients.

29. A pharmaceutical composition for preventing or treating a diabetes or an insulin-resistance syndrome, comprising a CGL1 polypeptide with a SEQ ID No 1 sequence, in association with one or more physiologically compatible excipients.

30. An antibody raised against a polypeptide according to any one of claims 23 to 25.

31. A method for detecting the presence of a polypeptide according to any one of claims 23 to 25 in a sample, comprising the following steps of:
a) contacting an antibody raised against a polypeptide according to any one of claims 23 to 25 with the sample to be tested;
b) detecting the optionally formed polypeptide-antibody complexes.

32. A set or kit for detecting a polypeptide according to any one of claims 23 to 25 in a sample, comprising:
a) an antibody raised against the polypeptide according to any one of claims 23 to 25;
b) if need be, the reactants required for detecting any optionally formed polypeptide-antibody complex.

33. A method for screening a candidate compound interacting with the CGL1 polypeptide with a SEQ ID No 1 sequence or with a fragment of the CGL1 polypeptide, characterized in that it comprises the following steps of:
a) contacting the candidate compound with the CGL1 polypeptide or the fragment of the CGL1 polypeptide;
b) detecting the optionally formed complexes between the CGL1 polypeptide or the fragment of the CGL1 polypeptide, on the one hand, and the candidate compound, on the other hand.

34. A set or kit for screening a candidate compound interacting with the CGL1 polypeptide with the SEQ ID No 1 sequence or a fragment of the CGL1 polypeptide, characterized in that it comprises:
a) a CGL1 polypeptide or a fragment of the CGL1 polypeptide;
b) if need be, the reactants required for detecting the optionally formed complexes between the CGL1 polypeptide and the fragment of CGL1 polypeptide, on the one hand, and the candidate compound, on the other hand.

35. A method for screening a candidate compound modulating the expression of the cgl1 gene, characterized in that it comprises the following steps of:
a) culturing a host cell expressing, naturally or after a genetic recombination, the cgl1 gene;
b) contacting the host cell cultured in step a) with a candidate compound;
c) determining the ability of the candidate compound to modulate the expression of the cgl1 gene by the host cell.

36. A set or kit for screening a candidate compound modulating the expression of the cgl1 gene, characterized in that it comprises:
a) a host cell expressing, naturally of after a genetic recombination, the cgl1 gene;
b) if need be, the means required for determining the ability of the candidate compound to modulate the expression of the cgl1 gene by the host cell.

37. A set or kit according to claim 36, characterized in that the means required for determining the ability of the candidate compound to modulate the expression of the cgl1 gene by the host cell are one or more probes specific for the cgl1 gene.

38. A non human transgenic animal having its somatic and/or germinal cells being transformed by a un nucleic acid according to claim 1.

39. A non human transgenic animal having its somatic and/or germinal cells being transformed by a nucleic acid being inserted into the genome so as to inactivate the gene corresponding to cgl1, in said transgenic animal.

40. A method for in vivo screening a candidate molecule or a substance modulating the expression of a nucleic acid according to any one of claims 1 to 5 comprising the following steps of:
a) administering the candidate substance or molecule to a non-human transgenic animal having its somatic and/or germinal cells transformed by an un nucleic acid according to any one of claims 1 to 5;
b) detecting the expression level of the nucleic acid according to any one of claims 1 to 5;
c) comparing the results obtained in b) with the results obtained in a transgenic animal which has not received the candidate substance or molecule.

41. A kit or set for in vivo screening a candidate molecule or substance modulating the expression of a nucleic acid according to any one of claims 1 to 5 comprising:
a) a non-human transgenic animal having its somatic and/or germinal cells transformed by a un nucleic acid according to any one of claims 1 to 5;
b) if need be, means for detecting the expression level of the nucleic acid according to any one of claims 1 to 5.

42. A method for in vivo screening a candidate molecule or substance modulating the expression or the activity of the CGL1 polypeptide, comprising the steps consisting of:
a) administering the candidate substance or molecule to a non-human transgenic animal having its somatic and/or germinal cells transformed by an un nucleic acid according to any one of claims 1 to 5 or a non-human transgenic animal having its somatic and/or germinal cells transformed by a nucleic acid inserted into the genome so as to inactivate the gene corresponding to cgl1, in said transgenic animal;
b) detecting the expression location and level of the CGL1 polypeptide using an antibody raised against the polypeptide wherein the polypeptide is the CGL1 polypeptide with a SEQ ID No. 1 sequence or a fragment or a variant of the CGL1 polypeptide, such a polypeptide wherein the variant of the CGL1 polypeptide is selected amongst the polypeptides with amino acid sequences from SEQ ID No. 2 to SEQ ID No. 10, or such a polypeptide wherein the fragment of the CGL1 polypeptide is a member selected from the group consisting of those having an amino acid sequence from SEQ ID No. 11 to SEQ ID No. 13; and
c) comparing the obtained results with those obtained in an animal which has not received the candidate substance or molecule.

43. A kit or set for in vivo screening a candidate molecule or substance modulating the expression or the activity of the CGL1 polypeptide, comprising:
a) a transgenic animal according to any one of claims 38 and 39;
b) if need be, the means for detecting the expression location or level of the CGL1 polypeptide.
